(19) United States
(12) Patent Application Publication
(10) Pub. No.: US 2020/0239398 A1
(43) Pub. Date: Jul. 30, 2020

(54) METHYLENE-CYCLOALKYLACETATE DERIVATIVES AND THEIR USE IN TREATMENT OF NEUROTROPIC CONDITIONS

(71) Applicant: Yissum Research Development Company of The Hebrew University of Jerusalem Ltd., Jerusalem (IL)

(72) Inventors: Dmitry TSVELIKHOVSKY, Nof Hagalil (IL); Philip LAZAROVICI, Jerusalem (IL); Dikla HAHAM, Jerusalem (IL); David LANKRI, Beit Shean (IL)

(73) Assignee: Yissum Research Development Company of The Hebrew University of Jerusalem Ltd., Jerusalem (IL)

(21) Appl. No.: 16/714,746

(22) Filed: Dec. 15, 2019

Related U.S. Application Data
(60) Provisional application No. 62/781,624, filed on Dec. 19, 2018.

(51) Int. Cl. C07C 57/26; A61P 25/28; C07D 211/70; C07D 309/26; C07D 335/02; C07C 59/82; C07C 255/31

(52) U.S. Cl. CPC C07C 57/26; A61P 25/28; C07D 211/70; C07C 255/31; C07D 335/02; C07C 59/82; C07D 309/26

(57) ABSTRACT

Methylene-cycloalkylacetate compounds and derivatives thereof and their use in methods for treatment of neurotropic conditions.

### BACKGROUND OF THE INVENTION

Neurotrophic factors such as nerve growth factor (NGF) play important roles in survival of neurons, differentiation of neuronal stem cells and neuronal regeneration.¹ Therefore, their activities might be beneficial in the therapy of neurodegenerative diseases and nerve regeneration.² However, the protein nature, the inability to cross the brain-blood barrier and high molecular weight properties restrict their clinical applications owing to their decreased bioavailability and unfavorable pharmacokinetics.³ Development of natural or synthetic neurotrophic factors mimetic drugs, which prevent or delay cell death and preserve or induce neuronal pathways by stimulating formation of axons, dendrites and synaptic connections is an unmet clinical need.⁴ Therefore, development of neurotropic drugs, which can induce neuronal sprouts regeneration, may represent an important development in regenerative medicine.⁵ The continuing screening of novel neuroprotective, neurotrophic (promoting survival) and neurotropic (promoting neurite outgrowth) drugs by pharmaceutical companies, utilizes synthetic chemical platforms followed by screening to discover an effective lead compound.⁶

### SUMMARY OF THE INVENTION

The inventors of the present invention have found methylene-cycloalkylacetate (MCA)-based compounds having neurotropic activity or NGF mimetic activity (resembling the activity of NGF), that can be used in the treatment to neurological conditions, such as for example brain disorders caused by insufficient trophic support, tissue engineering and regenerative medicine for neural regeneration.

Thus, in the first aspect of the present invention, there is provided a compound having the general formula (I), including any stereoisomer or salt thereof:

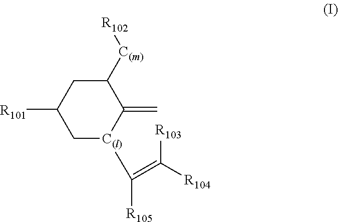

wherein
R₁₀₁ is selected from H, straight or branched C₁-C₁₀ alkyl, straight or branched C₂-C₁₀ alkenyl, straight or branched C₂-C₁₀ alkynyl;
m is an integer selected from 1-10; —C₍ₘ₎— is selected from a straight or branched alkylene, straight or branched alkenylene, straight or branched alkynylene; optionally interrupted by at least one heteroatom;
R₁₀₂ is selected from —C(═O)R₁₀₈, —C(═S)R₁₀₉, —C(═P)R₁₁₀, —C(═CR₁₁₁R₁₁₂)R₁₁₃, straight or branched C₂-C₁₀ alkenyl, straight or branched C₂-C₁₀ alkynyl;
each of R₁₀₈, R₁₀₉, R₁₁₀, R₁₁₁, R₁₁₂ and R₁₁₃ are independently is selected from a group consisting of OH, —OR₁₁₄, —NH₂, —NHR₁₁₅, —NR₁₁₆R₁₁₇;
each of R₁₁₄, R₁₁₅, R₁₁₆ and R₁₁₇ are independently selected from straight or branched C₁-C₁₀ alkyl;
l is an integer selected from 1-10; —C₍ₗ₎— is selected from a straight or branched alkylene, straight or branched alkenylene, straight or branched alkynylene; optionally interrupted by at least one heteroatom;
R₁₀₃, R₁₀₄, R₁₀₅ are each independently selected from H, straight or branched C₁-C₁₀ alkyl, C₂-C₁₀ alkenyl, C₂-C₁₀ alkynyl; provided that when R₁₀₁ is H: (i) at least one of R₁₀₃, R₁₀₄ and R₁₀₅ is different than H; OR (ii) m>1.

Thus, in some embodiments, at least one of R₁₀₃, R₁₀₄ and R₁₀₅ is different than H, therefore selected from straight or branched C₁-C₁₀ alkyl, C₂-C₁₀ alkenyl, C₂-C₁₀ alkynyl.

In other embodiments, the integer m is higher than 1, therefore in the range of 2-10.

In some embodiments, R₁₀₂ is a straight or branched C₁-C₁₀ alkyl. In other embodiments, R₁₀₂ is H.

In some embodiments, m is 1. In other embodiments, m is 2-5.

In some embodiments m is 2, l is 1 and R₁₀₂ is —C(═O)OH. In some embodiments m is 2, l is 1 and R₁₀₂ is —C(═O)OH and R₁₀₃, R₁₀₄, R₁₀₅ are each independently selected from H.

In some embodiments, R₁₀₁ is H. In other embodiments, R₁₀₁ is a straight or branched C₁-C₁₀ alkyl.

In some embodiments, R₁₀₃ is a straight or branched C₁-C₁₀ alkyl.

In some embodiments, R₁₀₄ is a straight or branched C₁-C₁₀ alkyl.

In some embodiments, R₁₀₅ is a straight or branched C₁-C₁₀ alkyl.

In some embodiments, R₁₀₃, R₁₀₄, R₁₀₅ are each independently a straight or branched C₁-C₁₀ alkyl. In some other embodiments, wherein R₁₀₃, R₁₀₄, R₁₀₅ are H.

In some embodiments, 1 is 1-5.

In some embodiments R₁₀₁ is H. In some embodiments R₁₀₁ is straight or branched C₁-C₁₀ alkyl. In some embodiments R₁₀₁ is a straight or branched C₂-C₁₀ alkenyl. In some embodiments R₁₀₁ is a straight or branched C₂-C₁₀ alkynyl.

In some embodiments —C₍ₘ₎— is a straight or branched C₁-C₁₀ alkylene. In some embodiments —C₍ₘ₎— is a straight or branched C₂-C₁₀ alkenylene. In some embodiments —C₍ₘ₎— is a straight or branched C₂-C₁₀ alkynylene. In some embodiments —C₍ₘ₎— is further interrupted by at least one heteroatom.

In some embodiments R₁₀₂ is —C(═O)R₁₀₈. In some embodiments R₁₀₂ is —C(═S)R₁₀₉. In some embodiments R₁₀₂ is —C(═P)R₁₁₀. In some embodiments R₁₀₂ is —C(═CR₁₁₁R₁₁₂)R₁₁₃. In some embodiments R₁₀₂ is straight or branched C₂-C₁₀ alkenyl. In some embodiments R₁₀₂ is straight or branched C₂-C₁₀ alkynyl.

In some embodiments R₁₀₈ is OH. In some embodiments R₁₀₈ is —OR₁₁₄. In some embodiments R₁₀₈ is —NH₂. In some embodiments R₁₀₈ is —NHR₁₁₅. In some embodiments R₁₀₈ is —NR₁₁₆R₁₁₇.

In some embodiments R₁₀₉ is OH. In some embodiments R₁₀₉ is —OR₁₁₄. In some embodiments R₁₀₉ is —NH₂. In some embodiments R₁₀₉ is —NHR₁₁₅. In some embodiments R₁₀₉ is —NR₁₁₆R₁₁₇.

In some embodiments R₁₁₀ is OH. In some embodiments R₁₁₀ is —OR₁₁₄. In some embodiments R₁₁₀ is —NH₂. In some embodiments R₁₁₀ is —NHR₁₁₅. In some embodiments R₁₁₀ is —NR₁₁₆R₁₁₇.

In some embodiments —C₍ₗ₎— is a straight or branched C₁-C₁₀ alkylene. In some embodiments —C₍ₗ₎— is a straight or branched C₂-C₁₀ straight or branched alkenylene. In some embodiments —C₍ₗ₎— is a straight or branched C₂-C₁₀ straight or branched alkynylene. In some embodiments each of —C₍ₗ₎— is further interrupted by at least one heteroatom.

In some embodiments, the compounds is selected from the following:

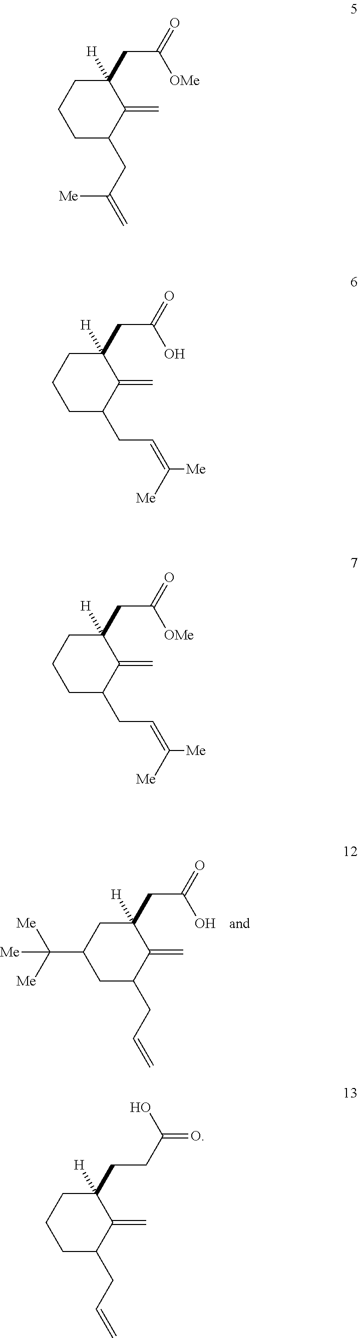

In another aspect the invention provides the compounds as disclosed hereinabove for use in the treatment of a neurotropic condition.

The term “neurotropic condition” refers to any condition that involves the degeneration and injury of the brain and peripheral nervous system neurons. Thus, the compounds of the present invention treat such conditions by promoting neuronal survival, proliferation and differentiation.

Thus, the compounds of the invention are defined as neuroprotective agents (neurotrophic agents (promoting neuronal survival), neurotropic agents (promoting neurite outgrowth), neuroplasticity enhancers (promoting neuronal differentiation), nootropic agents (enhancing cognition) and so forth). The compounds of the invention can be used in the treatment of many central nervous system neurological diseases such as Parkinson's disease, Alzheimer's disease, traumatic brain injuries, stroke and psychiatric disorders among others. The compounds of the invention can be used in the treatment of neurodegenerative diseases.

In some embodiments, said neurotropic condition is associated with neuronal cell death and/or loss of neuronal pathways.

In other embodiments, said neurotropic condition benefits from mimicking Neuronal Growth Factor (NGF) activity.

In a further aspect the invention provides the compounds as disclosed herein above, for use in peripheral nerve repair.

When referring to “peripheral nerve repair” it should be understood to encompass the neuroprotective effect of the compounds of the invention wherein neurite outgrowth (axonal regeneration) is promoted, the number of cells bearing neurites is increased, the length of primary neurites is increased and sprouting of neurite-like processes is induced. The compounds of the invention thus provide advanced nerve guidance channels (NGCs) acting as physical guidance for regeneration of nerves across lesions. NGCs containing the compounds of the invention present multifunctional properties aiming to direct the sprouting of axons from the proximal nerve end and to induce axonal and dendrite outgrowths obligatory for functional nerve regeneration thus avoiding or minimizing end-organ (e.g. muscle) degeneration.

In some embodiments, said neurotropic condition is selected from Parkinson's disease, Alzheimer's disease, traumatic brain injuries, stroke, psychiatric disorders, choreas, ALS, conditions benefiting from neuronal regeneration, conditions benefiting from scaffolds for neural tracks regeneration, conditions benefiting from neural stem cell differentiation, conditions benefiting from tissue engineering, conditions benefiting from regenerative medicine and any combinations thereof.

In another one of its aspects the invention provides a compound of the general formula (II), including any stereoisomer or salt thereof, for use in the treatment of a neurotropic condition:

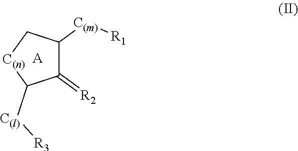

wherein
Ring A is optionally a saturated or unsaturated ring having optionally at least one heteroatom; and is optionally substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen;
R₄, R₅ and R₆ are each independently selected from H, halogen, straight or branched C₁-C₁₀ alkyl, straight or branched C₂-C₁₀ alkenyl, straight or branched C₂-C₁₀ alkynyl;
R₇ is selected from H, straight or branched C₁-C₁₀ alkyl, straight or branched C₂-C₁₀ alkenyl, straight or branched C₂-C₁₀ alkynyl, halogen, OH, O(C₁-C₁₀)alkyl, NH₂, amine;
n is an integer selected from 1-10;
—C₍ₙ₎— is selected from a straight or branched alkylene, straight or branched alkenylene, straight or branched alkynylene; optionally interrupted by at least one heteroatom;
m is an integer selected from 1-10;
—C₍ₘ₎— is selected from a straight or branched alkylene, straight or branched alkenylene, straight or branched alkynylene; optionally interrupted by at least one heteroatom;
R₁ is selected from —C(═O)R₈, —C(═S)R₉, —C(═P)R₁₀, —C(═CR₁₁R₁₂)R₁₃, straight or branched C₂-C₁₀ alkenyl, straight or branched C₂-C₁₀ alkynyl;
each of R₈, R₉, R₁₀, R₁₁, R₁₂ and R₁₃ are independently is selected from a group consisting of OH, —OR₁₄, —NH₂, —NHR₁₅, —NR₁₆R₁₇;
each of R₁₄, R₁₅, R₁₆ and R₁₇ are independently selected from straight or branched C₁-C₁₀ alkyl;
R₂ is selected from O, S, CR₁₈R₁₉;
each of R₁₈ and R₁₉ is independently selected from H, straight or branched C₁-C₁₀ alkyl, halogen, CF₃SO₃, OH, C₁-C₁₀ alkoxy.
l is an integer selected from 1-10; —C₍ₗ₎— is selected from a straight or branched alkylene, straight or branched alkenylene, straight or branched alkynylene; optionally interrupted by at least one heteroatom;
R₃ is selected from C(═O)R₂₀, OR₂₁, C(═O)OR₂₂, CF₃SO₃, straight or branched C₂-C₁₀ alkenyl, straight or branched C₂-C₁₀ alkynyl; each of alkenyl or alkylenyl groups are optionally substituted by at least one group selected from C(═O)R₂₃, OR₂₄, halogen, CF₃SO₃;
each of R₂₀, R₂₁, R₂₂, R₂₃ and R₂₄ is independently selected from H, OH, halogen, straight or branched C₁-C₁₀ alkyl, straight or branched C₁-C₁₀ alkoxy, NH₂, amine.

In some embodiments Ring A is a saturated 5, 6, 7, or 8 member ring (thus the ring consists of 5, 6, 7 or 8 atoms connected to each other with saturated single bonds only). In other embodiments Ring A is an unsaturated 5, 6, 7 or 8 member ring (thus the ring comprises at least one unsaturated bond within the ring structure. Said unsaturated bond can be a double and/or a triple bond between any two atoms in the ring). In further embodiments Ring A is a 5-7 member ring having at least one heteroatom (thus said ring comprises at least one atom that is different than carbon being selected from O, N or S at any position in the ring. When valency permits heteroatom is substituted with one or more H, straight or branched C₁-C₁₀ alkyl, straight or branched C₂-C₁₀ alkenyl, straight or branched C₂-C₁₀ alkynyl.

In some embodiments Ring A is a saturated ring. In other embodiments Ring A is an unsaturated ring. In some embodiments Ring A is further comprises at least one heteroatom. In other embodiments Ring A is substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen.

In some embodiments —C₍ₙ₎— is a straight or branched C₁-C₁₀ alkylene. In some embodiments —C₍ₙ₎— is a straight or branched C₂-C₁₀ alkenylene. In some embodiments —C₍ₙ₎— is a straight or branched C₂-C₁₀ alkynylene. In some embodiments —C₍ₙ₎— is further interrupted by at least one heteroatom.

In some embodiments —C₍ₘ₎— is a straight or branched C₁-C₁₀ alkylene. In some embodiments —C₍ₘ₎— is a straight or branched C₂-C₁₀ alkenylene. In some embodiments —C₍ₘ₎— is a straight or branched C₂-C₁₀ alkynylene. In some embodiments —C₍ₘ₎— is further interrupted by at least one heteroatom.

In some embodiments R₁ is —C(═O)R₈. In some embodiments R₁ is —C(═S)R₉. In some embodiments R₁ is —C(═P)R₁₀. In some embodiments R₁ is —C(═CR₁₁R₁₂)R₁₃. In some embodiments R₁ is straight or branched C₂-C₁₀ alkenyl. In some embodiments R₁ is straight or branched C₂-C₁₀ alkynyl.

In some embodiments R₈ is OH. In some embodiments R₈ is —OR₁₄. In some embodiments R₈ is —NH₂. In some embodiments R₈ is —NHR₁₅. In some embodiments R₈ is —NR₁₆R₁₇.

In some embodiments R₂ is O. In some embodiments R₂ is S. In some embodiments R₂ is CR₁₈R₁₉.

In some embodiments —C₍ₗ₎— is a straight or branched C₁-C₁₀ alkylene. In some embodiments —C₍ₗ₎— is a straight or branched C₂-C₁₀ alkenylene. In some embodiments —C₍ₗ₎— is a straight or branched C₂-C₁₀ alkynylene. In some embodiments —C₍ₗ₎— is further interrupted by at least one heteroatom.

In some embodiments R₃ is C(═O)R₂₀. In some embodiments R₃ is OR₂₁. In some embodiments R₃ is C(═O)OR₂₂. In some embodiments R₃ is CF₃SO₃. In some embodiments R₃ is straight or branched C₂-C₁₀ alkenyl. In some embodiments R₃ is straight or branched C₂-C₁₀ alkynyl. In some embodiments each of said alkenyl or alkylenyl groups of R₃ is further substituted by at least one group selected from C(═O)R₂₃, OR₂₄, halogen, CF₃SO₃.

The term “—C₍ₙ₎—” as used herein refers to a straight or branched hydrocarbon chain that can be saturated (i.e. having only single bonds connecting the atoms in the chain) or unsaturated (i.e. having at least one unsaturated bond, double or triple bond, connecting the atoms in the ring), having m carbon atoms. “—C₍ₙ₎—” chain can be optionally interrupted by at least one heteroatom, thus any two carbon atoms in the chain can be interrupted with at least one heteroatom between them (for example . . . —C—N—C—. . . ). Said heteroatom selected from O, N, S, P, when valency permits heteroatom is substituted with one or more H, straight or branched C₁-C₁₀ alkyl, straight or branched C₂-C₁₀ alkenyl, straight or branched C₂-C₁₀ alkynyl.

The term “—C₍ₘ₎—” as used herein refers to a straight or branched hydrocarbon chain that can be saturated (i.e. having only single bonds connecting the atoms in the chain) or unsaturated (i.e. having at least one unsaturated bond, double or triple bond, connecting the atoms in the ring), having m carbon atoms. “—C₍ₘ₎—” chain can be optionally interrupted by at least one heteroatom, thus any two carbon atoms in the chain can be interrupted with at least one heteroatom between them (for example . . . —C—N—C— . . . ). Said heteroatom selected from O, N, S, P, when valency permits heteroatom is substituted with one or more H, straight or branched C₁-C₁₀ alkyl, straight or branched C₂-C₁₀ alkenyl, straight or branched C₂-C₁₀ alkynyl.

In some embodiments, —C₍ₘ₎— is selected from a C₁-C₁₀ straight or branched alkylene, C₂-C₁₀ straight or branched alkenylene, C₂-C₁₀ straight or branched alkynylene. In some further embodiments, —C₍ₘ₎— is a C₁-C₁₀ straight or branched alkylene.

In some embodiments, R₁ is —C(═O)R₈. In other embodiments, R₁ is —C(═O)OR₁₄.

In some embodiments, R₂ is O. In some other embodiments, R₂ is CH₂.

The term “—C₍ₗ₎—” as used herein refers to a straight or branched hydrocarbon chain that can be saturated (i.e. having only single bonds connecting the atoms in the chain) or unsaturated (i.e. having at least one unsaturated bond, double or triple bond, connecting the atoms in the ring), having 1 carbon atoms. “—C₍ₗ₎—” chain can be optionally interrupted by at least one heteroatom, thus any two carbon atoms in the chain can be interrupted with at least one heteroatom between them (for example . . . —C—N—C— . . . ). Said heteroatom selected from O, N, S, P, when valency permits heteroatom is substituted with one or more H, straight or branched C₁-C₁₀ alkyl, straight or branched C₂-C₁₀ alkenyl, straight or branched C₂-C₁₀ alkynyl.

In some embodiments, —C₍ₗ₎— is selected from a C₁-C₁₀ straight or branched alkylene, C₂-C₁₀ straight or branched alkenylene, C₂-C₁₀ straight or branched alkynylene. In other embodiments, —C₍ₗ₎— is a C₁-C₁₀ straight or branched alkylene.

In some embodiments, R₃ is a straight or branched C₂-C₁₀ alkenyl optionally substituted by at least one group selected from —C(═O)R₂₀, OR₂₁, halogen and CF₃SO₃. In some further embodiments, R₃ is a straight or branched C₂-C₁₀ alkynyl optionally substituted by at least one group selected from —C(═O)R₂₀, OR₂₁, halogen and CF₃SO₃. In further embodiments, R₃ is —C(═O)R₂₀.

The term “treatment” as used herein refers to the administering of a therapeutic amount of a composition of the present invention comprising a compound of the present invention, which is effective to reduce, prevent or ameliorate undesired symptoms associated with the sensation of pain caused by any means (internal or external to the human body of a subject in need thereof).

The “effective amount” for purposes disclosed herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half-life in the body, on undesired side effects, if any, on factors such as age and gender, etc.

The pharmaceutical compositions of the invention may comprise additionally any other suitable substances such as other therapeutically useful substances, diagnostically useful substances, pharmaceutically acceptable carriers or the like.

When referring to “composition(s)” or “pharmaceutical composition(s)” the present invention seeks to include any compositions suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, flavouring agents, anti-oxidants, and wetting agents. Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration. The invention further includes a pharmaceutical composition, as hereinbefore described, in combination with packaging material, including instructions for the use of the composition for a use as hereinbefore described. For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

It is to be understood that the compounds provided herein may contain one or more chiral centers. Such chiral centers may be of either the (R) or (S) configuration. Thus, the composition of the invention may be enantiomerically pure (i.e. containing only a single enantiomer or diastereomer of the compound), or be stereoisomeric or diastereomeric mixtures.

The invention also includes any salt of a compound of formula (I), including any pharmaceutically acceptable salt, wherein a compound of the invention has a net charge (either positive or negative) and at least one counter ion (having a counter negative or positive charge) is added thereto to form said salt. The phrase “pharmaceutically acceptable salt(s)”, as used herein, means those salts of compounds of the invention that are safe and effective for pharmaceutical use in mammals and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. SCI. 1-19 (1977), incorporated herein by reference.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any integer or step or group of integers and steps.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows the neurotropic activity of MCAs of the invention at 10 μM after 2 and 7 days of treatment of PC12 cells.

FIGS. 2A-2G shows the light microscopy images of PC12 cells after 7 days of treatment with 10 μM of different MCA compounds of the invention (2A—Control, 2B—NGF, 2C—Compound 5, 2D—Compound 6, 2E—Compound 7, 2F—Compound 12, 2G—Compound 13). Magnification×100.

FIGS. 3A-3B shows the neurotropic effect analysis after overlaying the neurite outgrowths. FIG. 3A is the original photo of the neurotropic effect taken by the light microscope camera at a magnification of ×100; FIG. 3B is the overlaid neurite outgrowths skeletonized in a 0-225 gray scale by Image J in order to calculate D_{f}.

FIGS. 4A-4D. Analysis of different blood parameters of HU-MCA-13-injected mice (n=4, black) compared to DMSO treated group (n=3; white); 4A. Blood count; 4B. electrolytes; 4C. Coagulation; 4D. Kidney and liver functions. *p<0.01 compared to control; ALP; alkaline phosphatase; ALT, alanine transaminase; aPTT, activated partial thromboplastin time; AST, aspartate transaminase; BUN, blood urea nitrogen; INR, international normalized ratio; MCH, mean corpuscular hemoglobin; MCHC, mean corpuscular hemoglobin concentration; MCV, mean corpuscular volume; MPV, mean platelet volume; PT, prothrombin time; RBC, red blood cells; WBC, white blood cells.

FIG. 5 showing the additive neurotropic effect between HU-MCA-13 and NGF in the PC12 neuronal model.

FIGS. 6A-6I. HU-MCA-13 induced neurotropic effect in different neuronal cultures. Neurotropic effect was measured after treatment with 5 μM of HU-MCA-13 of rat PC12 dopaminergic neurons (7 days, 6A, 6B, 6C), mice dorsal root ganglion (DRG) explants (5 days, 6D, 6E, 6F) and rat spinal cord sensory neurons (14 days, 6G, 6H, 6I). The negative control cultures were treated with 0.1% DMSO and the positive control cultures were treated with 50 ng/ml mouse β-nerve growth factor (NGF) for the same periods of time. Neurite outgrowths are indicated by red arrows. The neurite outgrowth Df, or mean length (am) and the ratio of neurites/DRG area were measured from light microscopic photos. All data are expressed as the mean±SD. Statistical analysis was performed using one-way analysis of variance followed by Tukey's test. *p<0.01 compared to control.

FIGS. 7A-7D. HU-MCA-13 induced neurotropic effects in different neuronal cultures measured by expression of the neuronal axons marker βIII tubulin. 7A—PC12; 7B—SC neuron; 7C, 7D—DRG explant. 7A-7C—treatment with HU-MCA-13 (like FIG. 6); 7D—treatment with NGF (like FIG. 6). βIII tubulin was detected in the neuronal cultured cells using a mouse anti-neuron-specific βIII tubulin (clone TuJ-1) monoclonal antibody (R&D Systems, catalog number: MAB1195). The cells were stained in green.

FIGS. 8A-8C. Quantization of the neurotropic effect of HU-MCA-13. The neurotropic effect was quantified by evaluating macroscopically micrographs according to two parameters: length of neurite outgrowth, as determined by either the Df parameter for PC12 cells (8A) or measured with a micrometer scale for DRG (8B) and SCN neurons (8D) and the ratio between the neurites and DRG areas (8C); PC12 cells were treated for 2 or 7 days, DRG explants were treated for 5 days and SCN neurons were treated for 14 days. Control cultures were treated with 0.1% DMSO; NGF, represents cultures treated with 50 ng/ml mouse βNGF; Data are means±SD of at least 25 cells in three images each from three independent experiments.*p<0.05 by comparison to control.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

The compounds of the invention were shown to be able to regenerate neurotropic activity that could be used to treat brain neurological disorders and regenerative medicine. For instance, the neurotropic and NGF-enhanced effects were shown in PC12 model using natural herb products, diterpenenes from the plant Croton yanhuii, sesquiterpenes and iridoids from the plant Valeriana, sargaquinoic acid from the marine brown alga Sargassum macrocarpum, and prenylflavonoids abundant in many plants. Also, neurotropic and NGF enhancing effects were found in PC12 cells using synthetic molecules such as catecholamine precursor dihydroxyphenylalanine (L-DOPA), N-propargyl caffeate amide, pyrimidine heterocyclic compounds, and synthetic peptides.

A series of compounds 5-21 featuring diverse electronic and steric characteristics were prepared (displaying varying ring architectures and functional group combinations; Scheme 1).

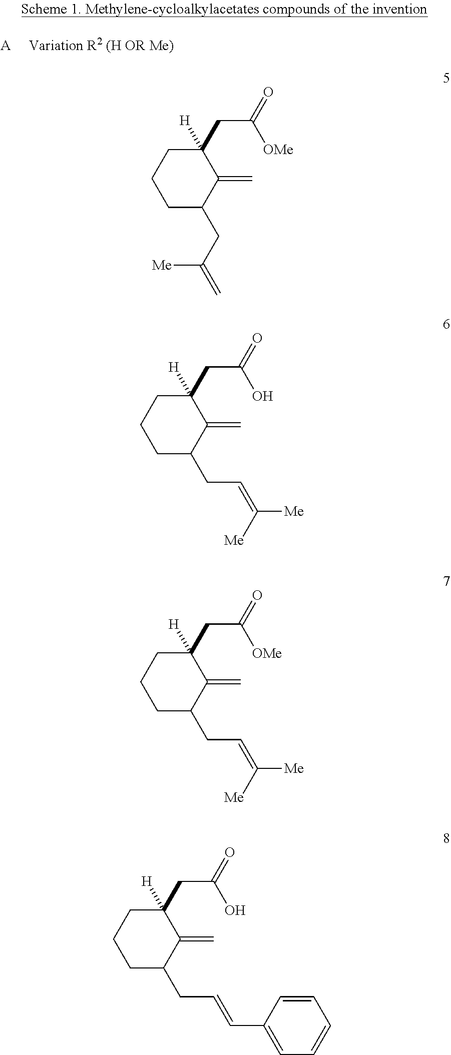

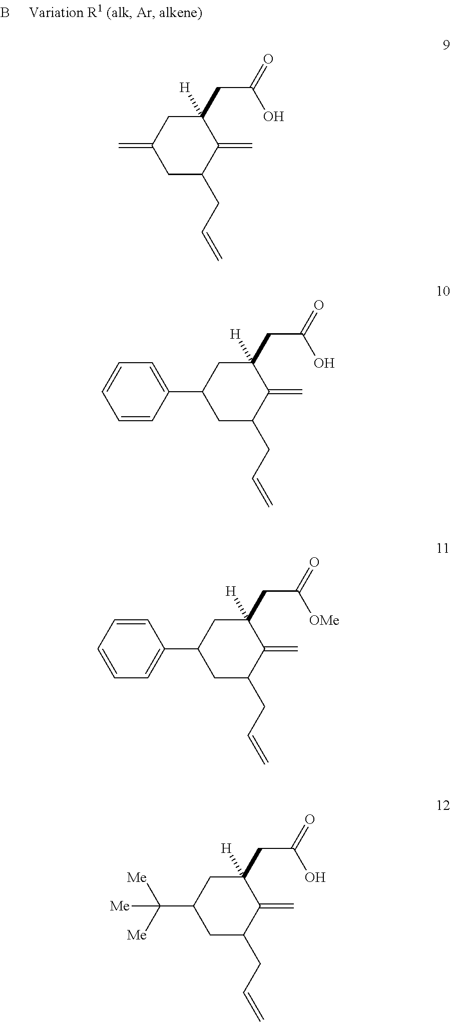

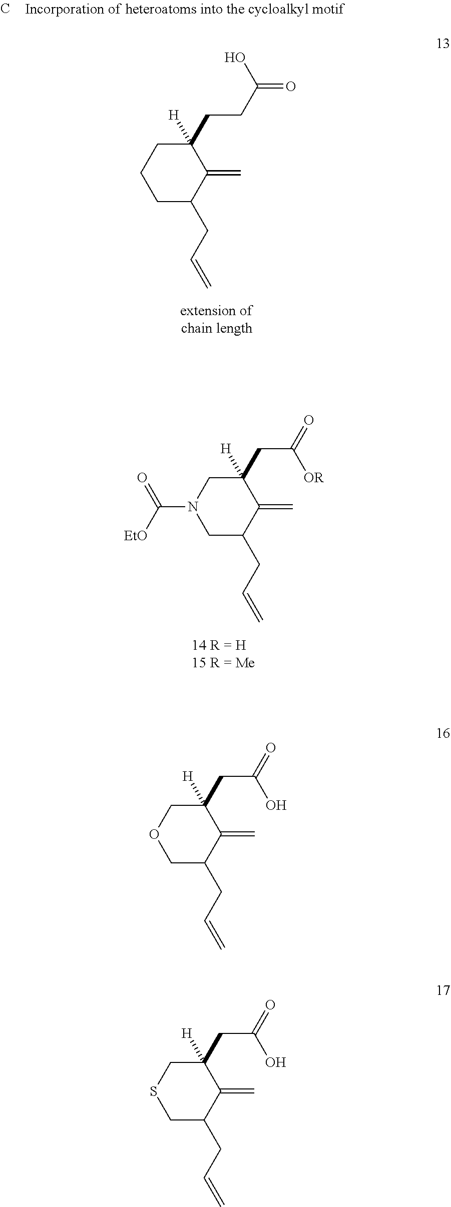

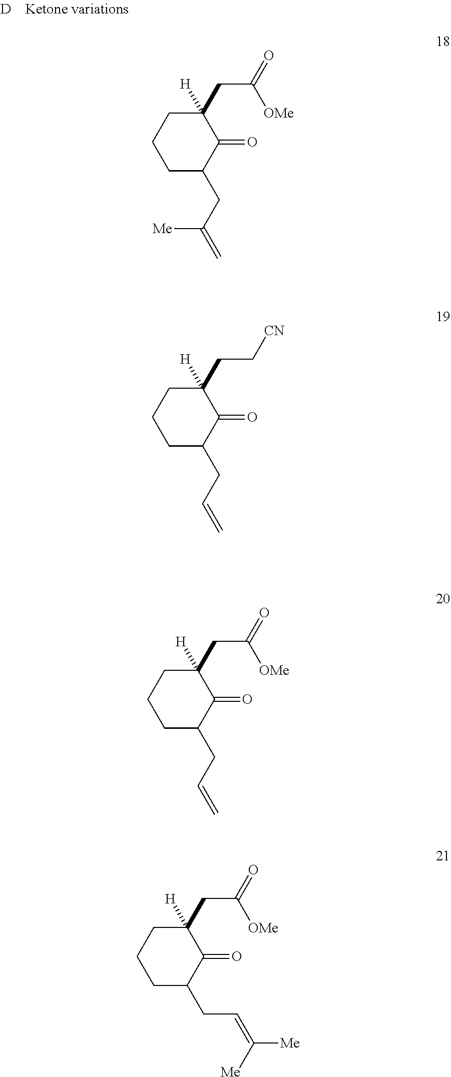

To examine whether the compact cycloalkyl scaffolds are endowed with neurotropic activity, the inventors have first screened a library of synthesized compounds 5-13, illustrated in Scheme 1. The functional modifications of general methylene-cycloalkylacetate frame, in this case, were performed on the alkene-bearing chains (5-8), the cyclic domains (9-12) and the length of an acid chain (compound 13).

Prior to investigation of the activation profile of the compounds, using the PC12 cell neurotropic assay, the inventors evaluated all compounds for cytotoxicity from 0.1 to 100 μM. Since the majority of compounds were not cytotoxic at 10 μM they were evaluated and compared at this concentration (Table 1).

TABLE 1
Neurotropic activity of the methylene-
cycloalkylacetate novel derivatives

| Neurotropic effect (mean ± SE) | |
|---|---|
| Two Days | Seven Days |

| | | NGF | | NGF |
|---|---|---|---|---|
| Compound ^{a} | D_{f} | (%) | D_{f} | (%) |
| 5 | 0.42 ± 0.01 | 91.3 | 0.20 ± 0.17 | 43.5 |
| 6 | 0.14 ± 0.15 | 30.4 | 0.20 ± 0.17 | 43.5 |
| 7 | 0.12 ± 0.14 | 26.1 | 0.20 ± 0.17 | 43.5 |
| 8^{b} | n.a | | n.a | |
| 9 | n.a | | 0.01 ± 0.01 | 2.2 |
| 10 | n.a | | n.a | |
| 11 | n.a | | 0.01 ± 0.01 | 2.2 |
| 12 | n.a | | 0.17 ± 0.16 | 37.0 |
| 13 | 0.39 ± 0.01 | 84.8 | 0.45 ± 0.01 | 97.8 |
| 14 | n.a | | n.a | |
| 15^{b} | n.a | | n.a | |
| 16 | n.a | | n.a | |
| 17 | n.a | | n.a | |
| 18^{b} | n.a | | n.a | |
| 19 | n.a | | n.a | |
| 20 | n.a | | n.a | |
| 21 | n.a | | 0.01 ± 0.01 | 2.2 |

^{a} All compounds were evaluated at a concentration of 10 μM; n.a—not active.
^{b}Partial toxicity of about 30% of the culture was observed at 10 μM.

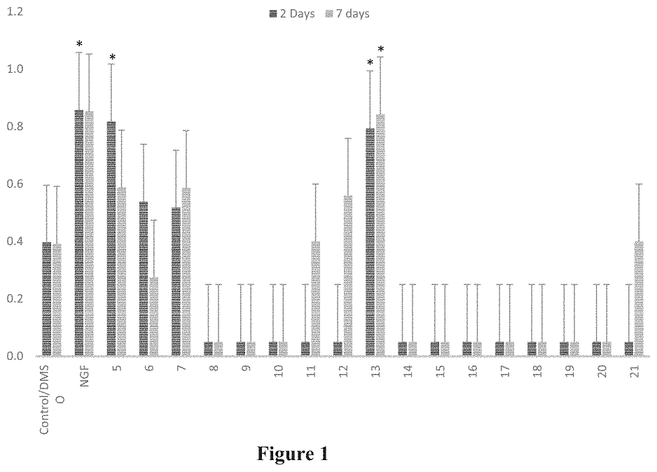

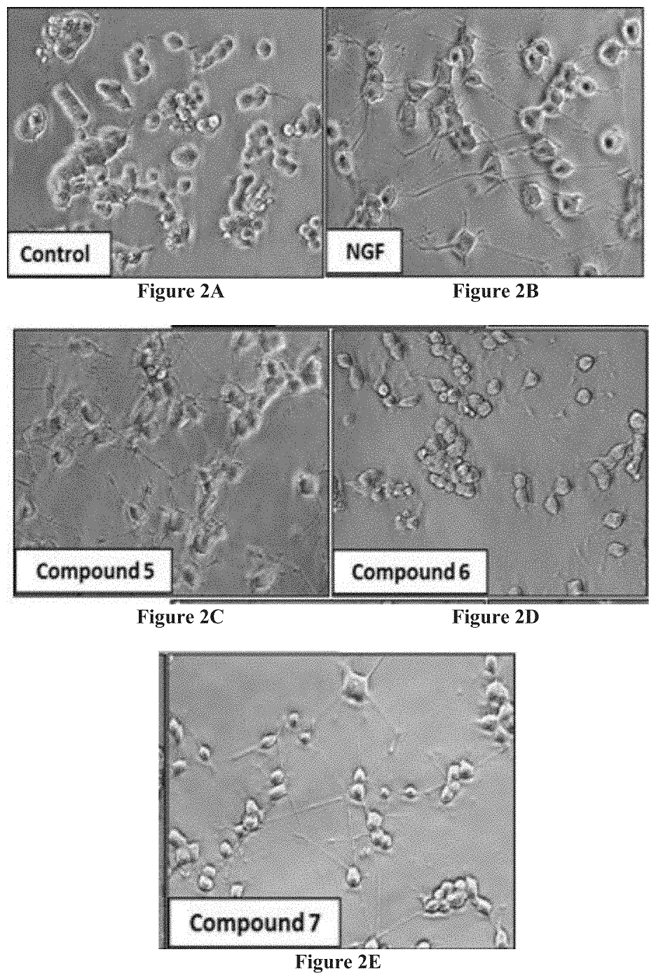

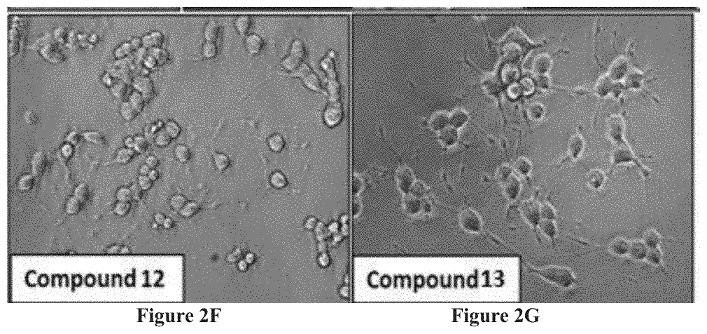

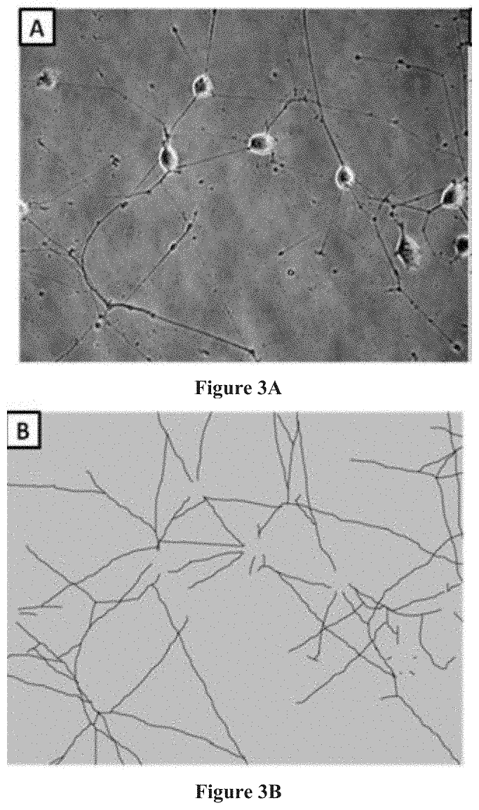

Compounds 5, 6, 7 and 13 were found the most active in inducing fast neurite outgrowth after two days of treatment, while after seven days of treatment a significant neurotropic activity was measured for compounds 5, 6, 7, 12 and 13 (Table 1 and FIGS. 1 and 2A-2G).

By comparing to NGF effect, the most potent compounds were 5>13>6>7 and 13>5, 6, 7>12 after two and seven days of treatment, respectively. Therefore, it was concluded that compounds 5 has a transient, acute effect on induction of neurite outgrowth, losing 50% activity from day two to seven, while for compounds 6, 7, 12 and 13, the neurotropic activity was gradually increased from day two to seven, reminiscent of NGF neurotropic activity time course. Thus, it was found that compact, methylene-cycloalkylacetate-based molecules could induce significant, NGF-like neurotropic activity, in PC12 neuronal cells. Interestingly, no activity was detected for compounds with substituted aromatic anchors (compounds 10 and 11), or 1,4-dimethylenated substrate (compound 9).

After concluding that structures 5, 6, 7, 12 and 13 are endowed with neurotropic activity, the inventors then examined the functionality of monocyclic variants integrated with heteroatoms. For this purpose, compounds 14-17 were designed according to the reported methodology, 21 and their neurotropic activity was identically measured using the PC12 cell neurotropic assay. As shown in FIG. 1 and Table 1, the incorporation of O, N or S atoms into the cyclic frame caused a complete loss of neurotropic activity.

Based on these results, a synthetic protocol was applied also to unsaturated variants of cycloalkylacetate substrates. For these series of molecules (compounds 18-21), the cyclic alkene group was substituted by a ketone residue, and their neurotropic activity was again measured using the PC12 cell neurotropic assay. No significant neurotropic activity was detected for the above-mentioned compounds. This observation suggests that an alkene element is indispensable for the neurotropic activity. However, for compounds 14-17, which resemble the topology of active compounds 5, 6, 7 and 12 and retain the double bond, an introduction of heteroatoms within the central ring resulted with complete loss of neurotropic activity. It should be noted that partial cytotoxic activity was detected for compounds 8, 15 and 18. Thus, due to interference with neurotropic activity, these MCA derivatives were excluded from the characterization.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Experimental Section

Unless otherwise stated, all reagents were purchased from commercial suppliers and used without further purification. DMEM medium, fetal calf (FCS) and horse (HS) serums, penicillin and streptomycin were purchased from Biological Industries (Beit Haemek, Afula Israel). Tissue culture grade mouse β-NGF, was purchased from Alomone Labs (Jerusalem, Israel). Solvents used in the reactions were distilled from appropriate drying agents prior to use. Reactions were monitored by thin-layer chromatography (TLC) on silica gel 60 F254 aluminium plates (Merck) and/or gas chromatography-mass spectrometry (GCMS). Visualization of compounds on TLC was accomplished by irradiation with UV light at 254 nm and/or vanillin stain. GCMS Analysis was performed with ‘Agilent 7820A’ gas chromatograph equipped with ‘Agilent 5975’ quadrupole mass selective detector, using Agilent HP-5MS capillary column (30 m, 0.25 mm, 0.25 μm film). Column chromatography was performed using silica gel 60 (particle size 0.040-0.063 mm) purchased from Sigma-Aldrich. Proton and carbon NMR spectra were recorded on Varian Mercury 300 MHz or Varian Mercury 500 MHz spectrometer in deuterated solvent. Proton chemical shifts are reported in ppm (å) relative to tetramethylsilane with the solvent resonance employed as the internal standard (CDCl₃, δ 7.26 ppm). ¹³C chemical shifts are reported in ppm from tetramethylsilane with the solvent resonance as the internal standard (CDCl₃, δ 77.0 ppm). Data are reported as follows: chemical shift, multiplicity (s=singlet, d=doublet, t=triplet, q=quartet, m=multiplet), integration and coupling constants (Hz). Infrared (IR) spectra were recorded on a Thermo Fischer Scientific NICOLET iS10 spectrometer.

Methods

Cell Cultures—

PC12 cells were grown in T-200 flasks in high glucose (4.5 gr/L) DMEM medium supplemented with 7% FCS, 7% horse serum and 1% penicillin and streptomycin. Cells were incubated at 37° C. in a humidified incubator containing 6% CO₂. All experiments were carried out in a clean room, according to ISO7 requirements (10,000 particles/m³).

Cell Seeding—

Tissue culture Falcon plates were coated with 200 μg/ml collagen type 1 and placed under UV light for 30 minutes for sterilization. Thereafter, one ml of PC12 cell suspension (5000 cells/well) was applied in 12 or 24 well plate, respectively. The cultures were grown in the incubator two days before exposure to investigated compound.

Neurotropic Activity (Neurite Outgrowth Assay)—

This experimental procedure contained two controls. The first consisted of untreated cells (negative control), representing the random effect: “noise signal”—the ability of cells to spontaneously grow neurite outgrowth which in seven days are of a length less than two-fold cell diameter. Also, negative controls consisted of cultures treated with 1% DMSO, solvent used to solubilize all compounds. The positive control consisted of cells treated with 50 ng/ml NGF, indicating maximal neurite outgrowth that can be achieved in this model. The experimental group consisted of PC12 cells treated with synthetic compounds. In each experiment, after two and seven days, six cultures were evaluated for neurotropic effect. These consecutive observations allow measurement of the progress of neurite outgrowth elongation and the percentage of cells with neurites.³¹

Analysis of the Neurotropic Activity—

In order to assess neurotropic effect, the neurite outgrowth length in each culture was quantified. For this purpose, each culture was placed under an inverted microscope and photographed by the attached camera. Each well was photographed at three to five representative areas. After acquiring the photos, they were analyzed by ImageJ, NIH software. The neurite outgrowth was estimated by fractal dimension (D_{f}), a statistical parameter that describes the fractional space (area and length) filled by neurite outgrowth. D_{f} ranged from 0 to 1.20. This method estimates the amount of picsel covered by neurites/cells compared to empty space per square field, and therefore plotting log (number of square boxes) vs. log (size in pixels) relationship generates a linear curve with D_{f} representing the slope of the curve. Every photograph that was taken, was opened in a Photoshop software and a new layer was generated on it. Using a 5-pixel wide digital pencil tool, all the outgrowths were marked and the layer with the markers (outgrowth network) was saved in a 0-255 gray scale as described in FIG. 2. Then, the saved layer was opened by ImageJ NIH software. The software “skeletonized” the layer, measured the length and complexity of every outgrowth in a fractal box count and calculated the fractional dimension parameter (D_{f}).³²

Cytotoxicity—

Cell death was evaluated by morphological appearance of the cells and release of LDH into the medium, in the absence and presence of different concentrations of synthetic compounds as previously described.³³

Statistics—

Each experiment was performed in duplicates and repeated three to four times (n=6-8). By using SPSS software, one way ANOVA was performed for the fractional dimension of each compound, in order to evaluate the neurotropic effect. In case of significance, Bonferroni post-hoc analysis was performed. The results were considered significant when p<0.05.

Acute Tolerance in Mice of HU-MCA-13 (Compound 13 in Scheme 1), Delivered by iv Injection in a Single-Dose

This pilot study was conducted to obtain acute information of the dose toxicity of HU-MCA-13 (Compound 13 in Scheme 1) in male mice. Male C57BL/6 mice, were injected intravenously with 0.2 ml of HU-MCA-13 in a dose of 250 mg/kg. Mice were monitored for 4 consecutive weeks for autonomic and neurotoxicity symptoms. The mortality and changes on body weight, clinical signs, gross observation, were monitored up to 30 days after treatment. No mortality was found at this high dose of 250 mg/kg. The results obtained in this study might predict that LD₅₀ after single dose of HU-MCA-13 must be over 2000 mg/kg in male mice. No significant changes on body weight were detected by comparing HU-MCA-13 250 mg/kg to 1% DMSO treated groups: they started with 21 gram body weight and ended with 27 gram body weight. The mice were examined once a week for autonomic symptoms by measuring salivation, urinary delivery, pupillary constriction, heart rate, blood pressure, and hair contraction. However no differences were observed between the HU-MCA-13 250 mg/kg to 1% DMSO treated groups. Occurrences of either flaccid or spastic paralysis of the legs were not observed. At injection of this high dose of 250 mg/kg body weight, the mice did not suffer from visible weakness and/or exhaustion. No paralysis, altered motor activity, or irregular behavior was observed in mice treated, suggesting a lack of neurotoxicity. Cutaneous hematomas around the injection or at distant locations site within 24 hours after injection have not been observed. Furthermore, no mice sudden deaths occurred within 24 hours after the injection or during the 4 weeks of observation. Sensory motor/neurological performance was weekly measured using several routine motor tests, but no neurological losses were observed (Table 2). Sensory motor tests were scored individually and according to the modified neurologic function severity score (mNSS). The data from each individual test and the total score calculated from individual tests that make up the mNSS were measured. The following assays were used: a. beam balance test—mice were placed on a 1-inch wide beam for 60 seconds. A normal response is balance with steady posture for 60 seconds (a score of 0). Deficits are scored if the rat: grasps the side of the beam (a score of 1), hugs the beam and one limb falls down from the beam (a score of 2), hugs the beam and two limbs fall off the beam (a score of 3), attempts to balance on the beam, but falls off from 60 seconds (a score of 4), attempts to balance on the beam but falls off from 60 seconds (a score of 5), or falls off with no attempt to balance or hang on the beam in <20 seconds (a score of 6); b. foot fault test—forelimb movement dysfunction, while walking on elevated metal grids, was monitored. The animal was placed on horizontal grids (85.5×26.5×20) cm with a glass enclosure for observation. With each weight bearing step, the forelimb can fall or slip between the metal support bars, an event recorded as a foot fault. The total number of forelimb steps and the total number of foot faults were recorded. The percentage of forelimb foot faults to the total steps that occurred within 2 minutes marks the test results; c. hind limb placing test—This test monitors reposition of hind limbs placed down and away from the table edge. The ability to retrieve and place the hind limbs back onto the table was scored. Immediate and complete limb retrieval was scored 0, delayed (>2 seconds) limb retrieval and/or interspersed flailing was scored 1, and no limb retrieval was scored 2; d. Increasing platform angle to slide test—This test monitors the strength and stability of resistance to slide down an inclined platform. It deploys a clean wooden board (60 cm by 40 cm) positioned horizontally. Mice were placed on the board with head facing up the increasing incline. The board then swings to increase the degree of inclination the rat experiences until slipping down the board feet first. The degree of the angle at which the rat relinquishes their grip/stance on the board (slips down) constitute the test results; e. the forelimb whole body suspension test—The test measures grip strength by duration of suspension by forepaws. The rat is suspended on a metal bar (diameter 5 mm) which it tightly holds onto by its forelimbs. The time (seconds) during which each rat could sustain its body weight while holding onto the bar is recorded. The mice drop from the bar onto soft material with no harm. The test was repeated three times and a mean result per rat was used as the score. Cumulatively these sensory motor/neurological findings indicate acute tolerability of 250 mg/kg HU-MCA-13 iv injection in Male C57BL/6 mice.

TABLE 2
Summary of sensory motor/neurological-neurobehavioral
tests results for each individual animal in the 5-day study

HU-MCA-13

| | |
|---|---|
| DMSO | (Compound 13 in Scheme 1) |

mNSS

| | | | | | | | |
|---|---|---|---|---|---|---|---|
| Day 0 | 0 | 0 | 0 | 0 | 0 | 0 | 0 |
| Day 1 | 0 | 0 | 0 | 0 | 0 | 0 | 0 |
| Day 5 | 0 | 0 | 0 | 0 | 0 | 0 | 0 |

Foot fault (%)

| | | | | | | | |
|---|---|---|---|---|---|---|---|
| Day 0 | 0/24 | 0/33 | 0/30 | 0/34 | 0/41 | 0/38 | 1/48 |
| Day 1 | 0/52 | 0/59 | 0/67 | 0/20 | 0/15 | 0/34 | 0/14 |
| Day 5 | 0/48 | 0/54 | 0/33 | 2/50 | 1/47 | 0/48 | 0/36 |

Hind limb

| | | | | | | | |
|---|---|---|---|---|---|---|---|
| Day 0 | 0 | 0 | 0 | 0 | 0 | 0 | 0 |
| Day 1 | 0 | 0 | 0 | 0 | 0 | 0 | 0 |
| Day 5 | 0 | 0 | 0 | 0 | 0 | 0 | 0 |

Beam balance

| | | | | | | | |
|---|---|---|---|---|---|---|---|
| Day 0 | 0 | 0 | 0 | 0 | 0 | 0 | 0 |
| Day 1 | 0 | 0 | 0 | 0 | 0 | 0 | 0 |
| Day 5 | 0 | 0 | 0 | 0 | 0 | 0 | 0 |

Self-suspension time (seconds)

| | | | | | | | |
|---|---|---|---|---|---|---|---|
| Day 0 | 10 | 7 | 8 | 8 | 10 | 9 | 10 |
| Day 1 | 8 | 14 | 10 | 14 | 12 | 15 | 12 |
| Day 5 | 9 | 14 | 13 | 13 | 13 | 15 | 14 |

Angle of increase (degrees)

| | | | | | | | |
|---|---|---|---|---|---|---|---|
| Day 0 | 35 | 35 | 35 | 35 | 35 | 35 | 35 |
| Day 1 | 35 | 35 | 35 | 35 | 35 | 35 | 35 |
| Day 5 | 35 | 35 | 35 | 35 | 35 | 35 | 35 |

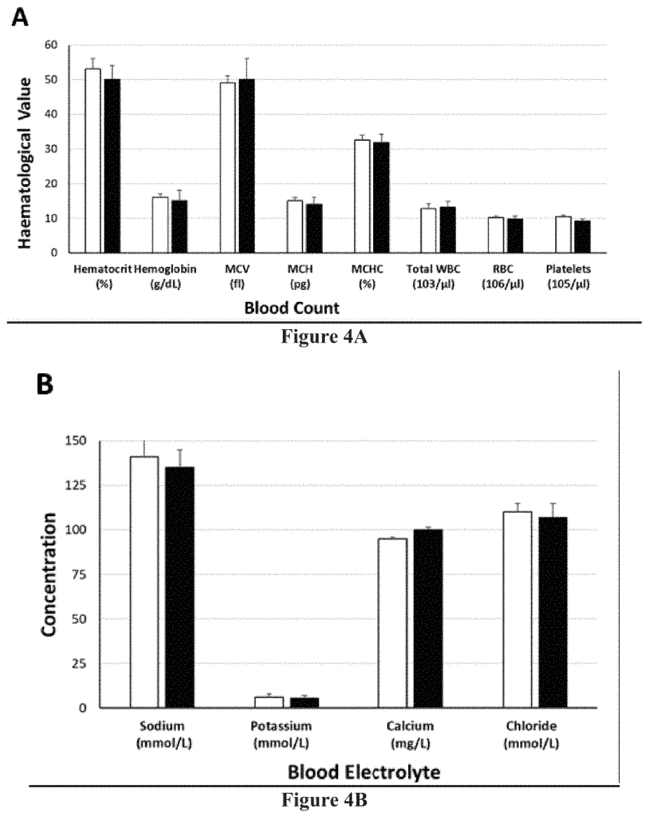

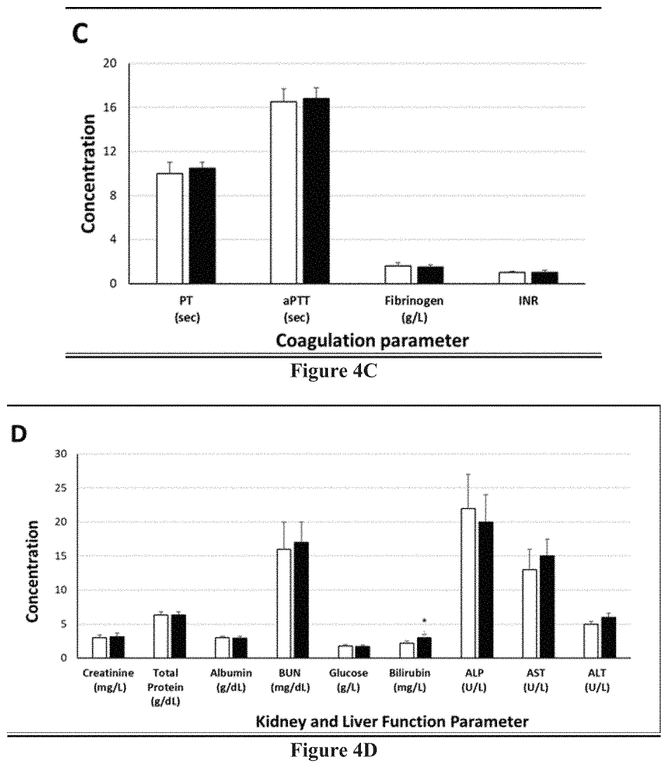

Tail vein blood samples were taken from DMSO 1% treated control (n=3) and HU-MCA-13-injected mice (n=4) after 24 hours from injection and submitted for hematocrit cell counting and biochemical analysis. Blood analyses are depicted in FIG. 4. No difference in any assay (blood count, electrolytes, coagulation parameters) was noted between the DMSO and HU-MCA-13-injected mice. Of special importance are normal liver (ALP, AST, ALT) functions and normal kidney (creatinine and blood urea nitrogen) functions, indicating sufficient organ integrity at the end of the experimental period. Moreover, coagulation biomarkers (International Normalized Ratio, prothrombin time, activated partial thromboplastin time, fibrinogen) were not affected by the exposure to HU-MCA-13. There was some fluctuation in both control and HU-MCA-13-injected mice on bilirubin concentration, which deserves further investigation. The data clearly indicates lack of differences on electrolytes and coagulation parameters between the two mice groups.

Cumulatively these blood analysis findings indicate acute tolerability of 250 mg/kg HU-MCA-13 upon iv injection in male C57BL/6 mice.

HU-MCA-13 Safety Profiling Using DiscoverX's SAFETY Scan In Vitro Pharmacological Profiling

On-target” activity refers to the site of action of the test substance (e.g., target receptor or enzyme) that results in the desired pharmacodynamic effect associated with the treatment of disease. “Off-target” activity refers to all other targets for which the molecule has affinity with the outcome of activation, blockade, or modulation, resulting in a functional effect. In many cases, the off-target activity of the molecule can be sub-clinical and not pose a concern. On the other hand, the off-target activity may result in side effects of the active agent that range from a minor nuisance to a severe adverse event in both preclinical studies and clinical trials. With this background, to further provide the basic information on the safety of HU-MCA-13 and exclude other potential molecular targets for toxicity, Safety74™ experiments were performed, to improve off-target liability testing. Safety47™ panel includes the receptors and kinases and other cellular targets recommended by major pharmaceutical companies for safety profiling of a novel drug entity. Assessing the specificity of HU-MCA-13 early in development, using highly relevant and predictive assays such as DiscoverX's SAFETYscan analysis, allows more informed decisions about compound safety, ultimately leading to the development of safer and more effective HU-MCA-13 drug.

In the analysis, 78 assays were performed utilizing the PathHunter enzyme fragment complementation (EFC) technology, FLIPR®-based cellular screening assays, KINOMEscan kinase binding assays, and a variety of enzymatic assays, accumulating 780 data points regarding the potential interaction of HU-MCA-13 with a large variety of in vitro pharmacological models of GPCR, tyrosine kinase receptors, second messenger and effector systems, using 10 μM of HU-MCA-13. This concentration was chosen since it is not toxic to neurons and since was found very active in stimulating neurite outgrowth in the dopaminergic PC12 cell culture neuronal model (original paper and patent).

Table 3, last column showing the maximal response measured with 10 μM HU-MCA-13, it is evident that HU-MCA 13 is not interacting with the majority of the targets with the exception of the 4 G-protein coupled receptors (GPCRs): α2A-adrenergic receptor ADRA2A (24.5%); Cannabinoid receptor CB1-CNR1 (50.1%) Cannabinoid receptor CB2-CNR2 (49.6%); Histamine H2 receptor HRH2 (26.9%) which are potential targets at high concentrations (doses) of HU-MCA-13. In such a case it is expected that upon using overdoses or toxic doses of HU-MCA-13 this could predict the physiological effects on blood pressure, adrenaline release, sedation, GI motility (ADRA2 related effects), euphoria and dysphoria; anxiety, memory impairment and poor concentration; analgesia, hypothermia weight loss, emesis, depression (CNR1 effects); inflammation and bone mass (CNR2 effects) and gastric acid secretion, emesis, and positive heart inotropic effects (HRH2 effects).

Cumulatively HU-MCA-13 profiling using pilot DiscoverX's SAFETYscan indicate Off-target safety and predicts some toxic effects over the therapeutic window. These findings strongly support further drug development of HU-MCA-13 pharmacophore.

TABLE 3
Screening in vitro of HU-MCA-13 effects on selected GPCRs, ion channels, kinases,
nuclear hormone receptors, enzymes and neurotransmitter transporters.

| | | | | | | |
|---|---|---|---|---|---|---|
| | | | | | | HU- |
| | | | | | | MCA-13 |
| Target | Target | Reference | | | | Maximal |
| Protein | Gene | Compound | Mode of | Functional | RC₅₀ | Response |
| name | name | Name¹ | action² | Assay³ | (μM)⁴ | (%)⁵ |

GPCRs

| | | | | | | |
|---|---|---|---|---|---|---|
| Adenosine | ADORA2A | NECA | Agonist | Calcium | 0.01524 | 0 |
| Receptor A_{2A} | | SCH 442416 | Antagonist | Flux | 0.04332 | 4.09 |
| Adrenergic | ADRA1A | A61603 | Agonist | Calcium | 0.0011 | 1.12 |
| Receptor α_{1A} | | Tamsulosin | Antagonist | Flux | 0.00096 | 3.06 |
| Adrenergic | ADRA2A | UK 14304 | Agonist | cAMP | 0.00066 | 24.55 |
| Receptor α_{2A} | | Yohimbine | Antagonist | | 0.02617 | 0 |
| Adrenergic | ADRB1 | Isoproterenol | Agonist | cAMP | 0.00125 | 1.92 |
| Receptor β₁ | | Betaxolol | Antagonist | | 0.02278 | 9.29 |
| Adrenergic | ADRB2 | Isoproterenol | Agonist | cAMP | 0.0016 | 1.29 |
| Receptor β₂ | | ICI 118,551 | Antagonist | | 0.00211 | 8.23 |
| Arginine | AVPR1A | [Arg⁸]- | Agonist | Calcium | 0.00052 | 0.29 |
| Vasopressin | | Vasopressin | | Flux | | |
| Receptor 1_{A} | | SR49059 | Antagonist | | 0.0029 | 0 |
| Cholecystokinin | CCKAR | (Tyr[SO₃H]27) | Agonist | Calcium | 0.0002 | 1.56 |
| Receptor | | Cholecystokinin | | Flux | | |
| A | | Fragment 26-33 | | | | |
| | | Amide | | | | |
| | | SR 27897 | Antagonist | | 0.0298 | 0 |
| Muscarinic | CHRM1 | Acetylcholine | Agonist | Calcium | 0.02359 | 0 |
| acetylcholine | | chloride | | Flux | | |
| Receptor M₁ | | Atropine | Antagonist | | 0.00695 | 7.43 |
| Muscarinic | CHRM2 | Acetylcholine | Agonist | cAMP | 0.02725 | 11.68 |
| acetylcholine | | chloride | | | | |
| Receptor M₂ | | Atropine | Antagonist | | 0.01538 | 0 |
| Muscarinic | CHRM3 | Acetylcholine | Agonist | Calcium | 0.04944 | 0.13 |
| acetylcholine | | chloride | | Flux | | |
| Receptor M₃ | | Atropine | Antagonist | | 0.00612 | 8.81 |
| Cannabinoid | CNR1 | CP 55940 | Agonist | cAMP | 0.00012 | 50.22 |
| Receptor 1 | | AM251 | Antagonist | | 0.00466 | 0.57 |
| Cannabinoid | CNR2 | CP55940 | Agonist | cAMP | 0.00033 | 49.59 |
| Receptor 2 | | SR144528 | Antagonist | | 0.0491 | 4.11 |
| Dopamine | DRD1 | Dopamine | Agonist | cAMP | 0.17552 | 0 |
| Receptor D₁ | | SCH 39166 | Antagonist | | 0.0026 | 14.3 |
| Dopamine | DRD2S | Dopamine | Agonist | cAMP | 0.00321 | 0 |
| Receptor D₂ | | Risperidone | Antagonist | | 0.00589 | 0.51 |
| Endothelin | EDNRA | Endothelin 1 | Agonist | Calcium | 0.00036 | 0 |
| Receptor | | BMS 182874 | Antagonist | Flux | 0.63689 | 0 |
| Type A | | | | | | |
| Histamine | HRH1 | Histamine | Agonist | Calcium | 0.0155 | 0 |
| Receptor H₁ | | Mepyramine | Antagonist | Flux | 0.01549 | 1.05 |
| Histamine | HRH2 | Histamine | Agonist | cAMP | 1.01395 | 1.78 |
| Receptor H₂ | | Tiotidine | Antagonist | | 0.06593 | 26.94 |
| 5-Hydroxy | HTR1A | Serotonin | Agonist | cAMP | 0.01101 | 7.66 |
| tryptamine | | Hydrochloride | | | | |
| (Serotonin) | | Spiperone | Antagonist | | 0.08756 | 0 |
| Receptor 1_{A} | | | | | | |
| 5-Hydroxy | HTR1B | Serotonin | Agonist | cAMP | 0.0046 | 10.47 |
| tryptamine | | Hydrochloride | | | | |
| (Serotonin) | | SB 224289 | Antagonist | | 0.03853 | 0 |
| Receptor 1_{B} | | | | | | |
| 5-Hydroxy | HTR2A | Serotonin | Agonist | Calcium | 0.0061 | 1.13 |
| tryptamine | | Hydrochloride | | Flux | | |
| (Serotonin) | | Altanserin | Antagonist | | 0.01903 | 0 |
| Receptor 2_{A} | | | | | | |
| 5-Hydroxy | HTR2B | Serotonin | Agonist | Calcium | 0.00263 | 0 |
| tryptamine | | Hydrochloride | | Flux | | |
| (Serotonin) | | LY 272015 | Antagonist | | 0.00082 | 0.13 |
| Receptor 2_{B} | | | | | | |
| Opioid | OPRD1 | DADLE | Agonist | cAMP | 0.00012 | 6.34 |
| Receptor | | Naltriben | Antagonist | | 0.00064 | 0 |
| Delta ₁ | | | | | | |
| Opioid | OPRK1 | Dynorphin A (1- | Agonist | cAMP | 0.0481 | 7.89 |
| Receptor | | 17) | | | | |
| Kappa ₁ | | Nor- | Antagonist | | 0.0064 | 0 |
| | | Binaltorphimine | | | | |
| Opioid | OPRM1 | DAMGO | Agonist | cAMP | 0.00194 | 9.31 |
| Receptor Mu ₁ | | Naloxone | Antagonist | | 0.00552 | 0.38 |

Ion channels

| | | | | | | |
|---|---|---|---|---|---|---|
| Voltage- | CAV1.2 | Isradipine | Blocker | Ion | 0.0225 | 3.82 |
| gated L-type | | | | channel | | |
| calcium | | | | | | |
| channel | | | | | | |
| Gamma- | GABAA | Picrotoxin | Blocker | | 5.91692 | 7.74 |
| aminobutyric | | GABA | Opener | | 8.51412 | 3.68 |
| acid | | | | | | |
| Receptor A | | | | | | |
| Kv11.1, the | hERG | Astemizole | Blocker | | 0.07136 | 0 |
| alpha subunit | | | | | | |
| of a | | | | | | |
| potassium | | | | | | |
| ion channel | | | | | | |
| 5-Hydroxy- | HTR3A | Bemestetron | Blocker | | 0.00368 | 0.19 |
| tryptamine | | Serotonin | Opener | | 0.552217 | 0 |
| (Serotonin) | | Hydrochloride | | | | |
| Receptor 3_{A} | | | | | | |
| Kv7.1/ | KvLQT1/ | XE 991 | Blocker | | 2.63679 | 0.55 |
| KCNE | minK | ML-277 | Opener | | 6.71743 | 0 |
| Potassium | | | | | | |
| voltage- | | | | | | |
| gated | | | | | | |
| channel | | | | | | |
| Nicotinic | nAChR | Dihydro-AY- | Blocker | | 0.70659 | 5.95 |
| acetylcholine | (a4/b2) | erythroidine | | | | |
| Receptor- | | Nicotine | Opener | | 2.18712 | 0 |
| alpha-4 beta- | | | | | | |
| 2 | | | | | | |
| A | NAVI1.5 | Lidocaine | Blocker | | 41.01752 | 0 |
| tetrodotoxin- | | | | | | |
| resistant | | | | | | |
| voltage- | | | | | | |
| gated sodium | | | | | | |
| channel | | | | | | |
| N-methyl-D- | NMDAR | MK 801 | Blocker | | 0.05277 | 0 |
| aspartate | (1A/2B) | L-Glutamic Acid | Opener | | 0.96247 | 0 |
| (NMDA) | | | | | | |
| Glutamate | | | | | | |
| Receptor | | | | | | |
| 1_{A}/2_{B} | | | | | | |

Kinases

| | | | | | | |
|---|---|---|---|---|---|---|
| Insulin | INSR | BMS-754807 | Inhibitor | Binding | 0.0009 | 3.22 |
| Receptor | | | | | | |
| (tyrosine | | | | | | |
| kinase) | | | | | | |
| Lymphocyte | LCK | Gleevec | Inhibitor | | 0.07191 | 12.77 |
| Cell-Specific | | | | | | |
| Protein- | | | | | | |
| Tyrosine | | | | | | |
| Kinase (Src | | | | | | |
| family) | | | | | | |
| Rho | ROCK1 | Staurosporine | Inhibitor | | 0.00034 | 21.61 |
| Associated | | | | | | |
| Coiled-Coil | | | | | | |
| Containing | | | | | | |
| Protein | | | | | | |
| Kinase 1 | | | | | | |
| (serine- | | | | | | |
| threonine | | | | | | |
| kinase) | | | | | | |
| Vascular | VEGFR2 | SU-11248 | Inhibitor | | 0.00033 | 21.68 |
| endothelial | | | | | | |
| growth factor | | | | | | |
| receptor 2 | | | | | | |
| (KDR | | | | | | |
| tyrosine | | | | | | |
| kinase) | | | | | | |

Nuclear Hormone Receptors

| | | | | | | |
|---|---|---|---|---|---|---|
| Nuclear | AR | 6a- | Agonist | Nuclear | 0.00646 | 0 |
| Hormone | | Fluorotestosterone | | Hormone | | |
| | | | | Receptor | | |
| Androgen | | Geldanamycin | Antagonist | Translocation | 0.08646 | 5.27 |
| Receptor | | | | | | |
| Nuclear | GR | Dexamethasone | Agonist | | 0.10198 | 0 |
| Hormone | | Mifepristone | Antagonist | | 0.10906 | 2.28 |
| Glucocorticoid | | | | | | |
| Receptor | | | | | | |

Enzymes

| | | | | | | |
|---|---|---|---|---|---|---|
| Acetylcholinesterase | AChE | Physostigmine | Inhibitor | Enzymatic | 0.0282 | 4.27 |
| Cyclooxygenase | COX 1 | Indomethacin | Inhibitor | | 0.06281 | 24.99 |
| 1 | | | | | | |
| Cyclooxygenase | COX 2 | NS-398 | Inhibitor | | 0.07376 | 7.99 |
| 2 | | | | | | |
| Monoamine | MAOA | Clorgyline | Inhibitor | | 0.00217 | 1.86 |
| oxidase type | | | | | | |
| A | | | | | | |
| cGMP- | PDE3A | Cilostamide | Inhibitor | | 0.02113 | 3.21 |
| inhibited | | | | | | |
| cyclic | | | | | | |
| nucleotide | | | | | | |
| phosphodiesterase | | | | | | |
| 3_{A} | | | | | | |
| cAMP- | PDE4D2 | Cilomilast | Inhibitor | | 0.01549 | 0 |
| specific 3′,5′- | | | | | | |
| cyclic | | | | | | |
| phosphodiesterase | | | | | | |
| 4D₂ | | | | | | |

Catecholamine Transporters

| | | | | | | |
|---|---|---|---|---|---|---|
| Dopamine | DAT | GBR 12909 | Blocker | Transporter | 0.0076 | 0 |
| transporter | | | | | | |
| Norepinephrine | NET | Desipramine | Blocker | | 0.01089 | 3.53 |
| transporter | | | | | | |
| Serotonin | SERT | Clomipramine | Blocker | | 0.00878 | 15.45 |
| transporter | | | | | | |

¹Full chemical names of reference compounds: NECA, 5′-(N-Ethylcarboxamido)adenosine; SCH 442416, 2-(2-furyl)-7-[3-(4-methoxyphenyl)propyl]-7H-pyrazolo[4,3-e][1,2,4]triazolo[1,5-c]pyrimidin-5-amine; A61603, N-(5-(4,5-Dihydro-1H-imidazol-2-yl)-2-hydroxy-5,6,7,8-tetrahydronaphthalen-1-yl)methanesulfonamide hydrobromide; Tamsulosin, 5-(2-((2-Ethoxyphenoxy)ethyl)amino) propyl-2-methoxybenzenesulfonamide; UK 14304, 5-Bromo-N-(2-imidazolin-2-yl)-6-quinoxalinamine; Yohimbine, methyl (1S,15R,18S,19R,20S)-18-hydroxy1,3,11,12,14,15,16,17,18,19,20,21-dodecahydroyohimban-19-carboxylate; Isoproterenol, 4-[1-hydroxy-2-(propan-2-ylamino)ethyl]benzene-1,2-diol; Betaxolol, 1-[4-[2-(cyclopropylmethoxy)ethyl]phenoxyl-3-(propan-2-ylamino)propan-2-ol; Isoproterenol, 4-[1-hydroxy-2-(propan-2-ylamino)ethyl]benzene-1,2-diol; ICI 118551, (2R,3S)-1-[(7-methyl-2,3-dihydro-1H-inden-4-yl)oxy]-3-(propan-2-ylamino)butan-2-ol; hydrochloride.
²The mode of interaction with the biological target;
³The assay describing the major function of the biological target;
⁴Reference compound effective concentration 50%;
⁵HU-MCA-13 maximal response at a concentration of 10 μM.

The additive neurotropic effect between HU-MCA-13 and NGF in the PC12 dopaminergic neuronal model A variety of in vitro central and peripheral neuronal models has been developed to understand the mechanisms underlying the regenerative failure of axons, and to guide pre-clinical development of regeneration-promoting therapeutics. By utilizing a combination of several in vitro models, this could balance the speed, convenience, and mechanistic resolution of simpler models for future in vivo pharmacological investigations on therapeutic effect in polyneuropathies.

Most neuronal high content screening assays use neurite outgrowth in vitro as a surrogate measure for process extension in vivo. This is due to multiple reasons including: i. the time required for nerves in vivo to grow and reliably express axonal or dendritic protein markers (weeks to months) is much over the optimal assay duration of in vitro neurite outgrowth assays (1-7 days), and ii. axonal markers are typically restricted to the neurite at some distance away from the cell body, making it difficult to automatically connect traced processes to the correct cell body number.

Accordingly, assayed living neurons are typically visualized by microscopy and fixed neurons by immunostaining for tubulin or tau (MAP) cytoskeleton proteins, and growth of putative axons and dendrites is often assessed simultaneously. Neurite outgrowth assays are among the most commonly utilized phenotypic screens relevant to axon regeneration. They have been successfully used to identify small molecules that can promote regeneration in vivo (Al-Ali et al. Rational Polypharmacology: Systematically Identifying and Engaging Multiple Drug Targets To Promote Axon Growth. ACS Chem. Biol. 2015; 10:1939-51). Neurons are typically grown on an adherent substrate that may be coated with one or more neuronal matrix proteins. Permissive substrates such as collagen I and IV and laminin are used to more easily identify agents that stimulate neurite outgrowth since on these matrices the neurons extend neurites at a physiological rate, making the correct window for detecting stimulation of neurite outgrowth and axon regeneration. For single endpoint assays, neurite outgrowth is measured dynamically in live low-density cultures in a label-free setting, using light microscopy followed by image analysis and data mining.

The Method:

PC12 cells were cultured in T-200 flasks in high glucose (4.5 gr/L) DMEM medium supplemented with 7% FCS, 7% horse serum and 1% penicillin and streptomycin. Cells will be maintained at 37° C. in a humidified incubator containing 6% CO₂. All experiments were carried out in a GLP clean room, according to ISO 7 requirements (10,000 particles/m³). For neurotropic experiments, tissue culture Falcon plates were coated with 200 μg/ml collagen type 1 and placed under UV light for 30 minutes for sterilization. Thereafter, one ml of PC12 cell suspension (5000 cells/well) was applied in 12 or 24 well plate, respectively. The cultures were maintained in the incubator two days before exposure to investigated compound. Each neurotropic experiment contained two controls. The first consisted of untreated cells (negative control), representing the random effect: “noise signal”—the ability of cells to spontaneously grow neurite outgrowth which in seven days are of a length less than two-fold cell diameter. Also, negative controls consisted of cultures treated with 1% DMSO, solvent used to solubilize HU-MCA-13. The positive controls represented neuronal cultures treated with 50 ng/ml NGF, indicating maximal neurite outgrowth that can be achieved in this model. The experimental group consisted of PC12 cells treated with 10 μM HU-MCA-13. In each experiment, after two and seven days, six cultures will be evaluated for neurotropic effect. These consecutive observations allow measurement of the progress of neurite outgrowth elongation and the percentage of cells with neurites (Katzir et al. A quantitative bioassay for nerve growth factor, using PC12 clones expressing different levels of trkA receptors. J Mol Neurosci. 2002; 18(3):251-64). In order to assess neurotropic effect, the neurite outgrowth length in each culture was quantified. For this purpose, each culture was placed under an inverted microscope and photographed by the attached camera. Each well was photographed at three to five representative areas. After acquiring the photos, they were analyzed by ImageJ, NIH software. The neurite outgrowth was estimated by fractal dimension (D_{f}), a statistical parameter that describes the fractional space (area and length) filled by neurite outgrowth. D_{f} ranged from 0 to 1.20. This method estimated the amount of picsels covered by neurites/cells compared to empty space per square field, and therefore plotting log(number of square boxes) vs. log(size in pixels) relationship generated a linear curve with D_{f} representing the slope of the curve. Every photograph that was taken, was opened in a Photoshop software and a new layer was generated on it. Using a 5-pixel wide digital pencil tool, all the outgrowths were marked and the layer with the markers (outgrowth network) were saved in a 0-255 gray scale. Thereafter, the saved layer was opened by ImageJ NIH software. The software was “skeletonize” the layer, measuring the length and complexity of every outgrowth in a fractal box count and calculated the fractional dimension parameter (D_{f}) (Arien-Zakay et al. Quantitative assessment of neuronal differentiation in three-dimensional collagen gels using enhanced green fluorescence protein expressing PC12 pheochromocytoma cells. J Mol Neurosci. 2009; 37(3):225-37).

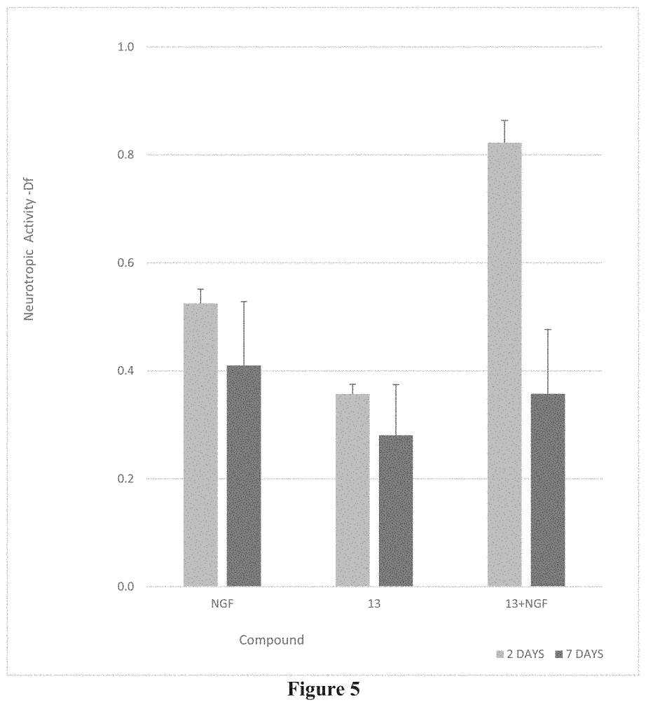

Using an established PC12 neurotropic in vitro assay, the regenerative enhancing effect of HU-MCA-13 when applied concomitantly, together with NGF, a known neurotrophin was investigated. It was found that HU-MCA-13 alone at 10 μM, enhanced neurite outgrowth to a D_{f} value of 0.36 at 2 days and 0.30 at 7 days of treatment. NGF alone, as positive control, at the conventional dose of 50 ng/ml enhanced neurite outgrowth to a D_{f} value of 0.54 at 2 days and 0.4 at 7 days of treatment (FIG. 5). To verify a possible additive effect, the cultures with a mixture of low dose NGF (1 ng/ml generating 10% neurite outgrowth=D_{f} of 0.05) and 1 μM HU-MCA-13 generating about 10-15% neurite outgrowth=D_{f} of 0.04) were tested and found after 2 days a D_{f} of 0.85 and after 7 days a D_{f} of 0.38. NGF is released by hematopoietic cells during polyneuropathy inflammatory process, this finding further strength the possibility of a potential enhancement regenerative effect of this drug upon in vivo therapy.

HU-MCA-13 Induced Neurite Outgrowth in Dorsal Root Ganglion (DRG) Sensory Neurons

Enhancing sensory axon regeneration after peripheral nervous system (PNS) and central nervous system (CNS) injury remains a goal of clinicians and scientists. Thus, being able to study the effect of HU-MCA-13 on sensory neuronal cultures obtained from dissociated dorsal root ganglia (DRG) has obvious benefits. A feature that distinguishes DRG neurons from other CNS neurons is that their peripheral axons can regenerate long distances and often re-establish appropriate functional connections after PNS injuries. Furthermore, these neurons become “pre-conditioned” during such a process, such that their central axons acquire an enhanced regenerative/sprouting response. A clear benefit of using DRG neurons is that all neurites extended from these cells are technically axons, as confirmed by immune staining for cytoskeletal-specific proteins. Furthermore, DRG neurons tend to extend neurites in culture at a much higher rate than other CNS neurons, which can shorten an assay's duration and compress its dynamic range. This is particularly true for neurite outgrowth assays.

To perform these experiments, about 10 dorsal root ganglia (DRGs) were excised under sterile conditions and transferred to 0.25% Type IV collagenase (Sigma-Aldrich-Merck) in Dulbecco's Modified Eagle Medium (DMEM) at 37° C. for 45 min, followed by a 20-min incubation with 0.025% trypsin in DMEM, to digest the associated connective tissue surrounding the neurons. After plating the ganglia onto collagen (50 μg/ml)/laminin mixture (5 μg/ml)-coated tissue culture wells for 24 h, DRG neurons were cultured for additional 1-7 days in fresh Neurobasal A/B27 containing either 50 ng/ml NGF (positive control), blank medium (DMSO 1%) control and HU-MCA-13. The cultures were photographed; thereafter the ganglia were immune-stained for neuronal tubulin and again photographed using a fluorescent microscope. Image analysis was performed by a researcher blinded to the experimental conditions. Neurite outgrowth distance measured in micrometers, was quantified as for PC12 cultures.

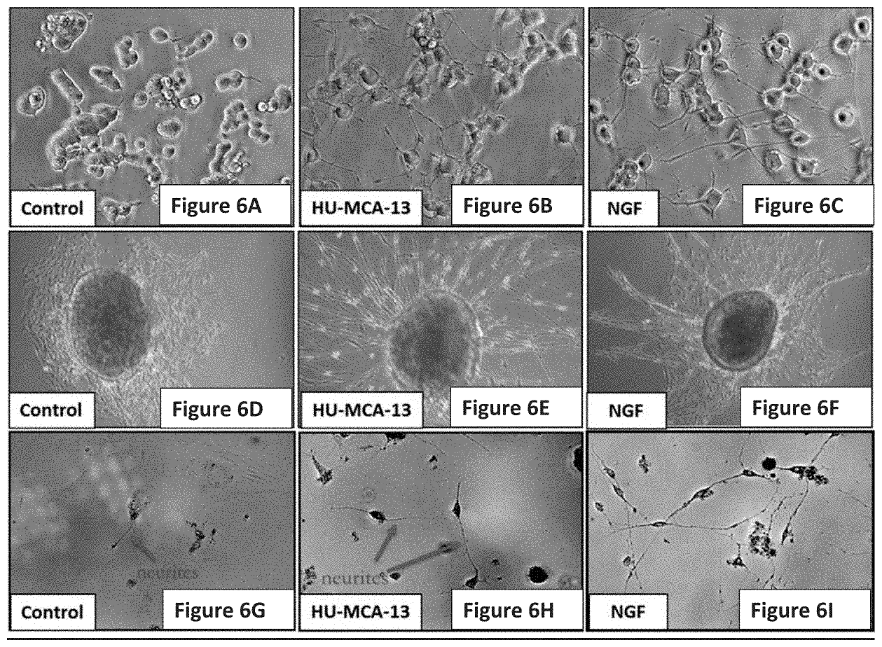

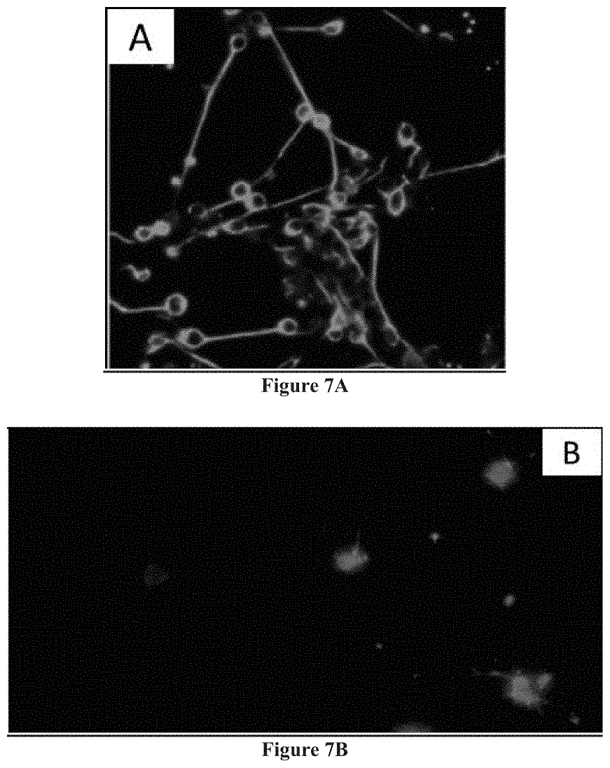

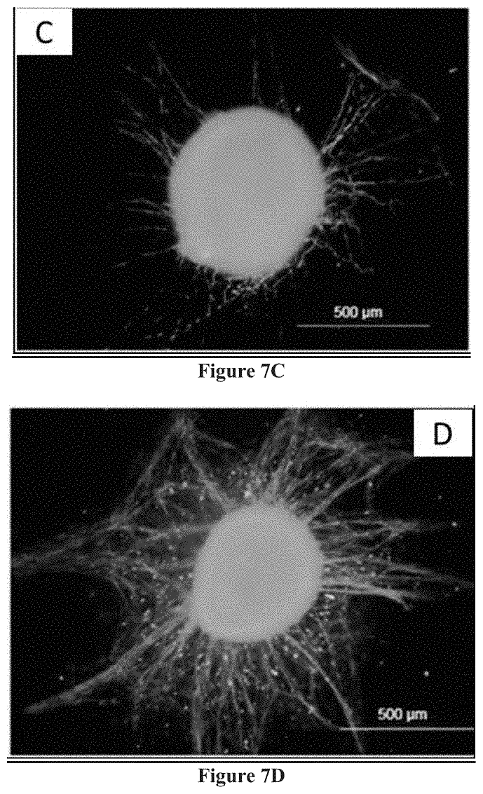

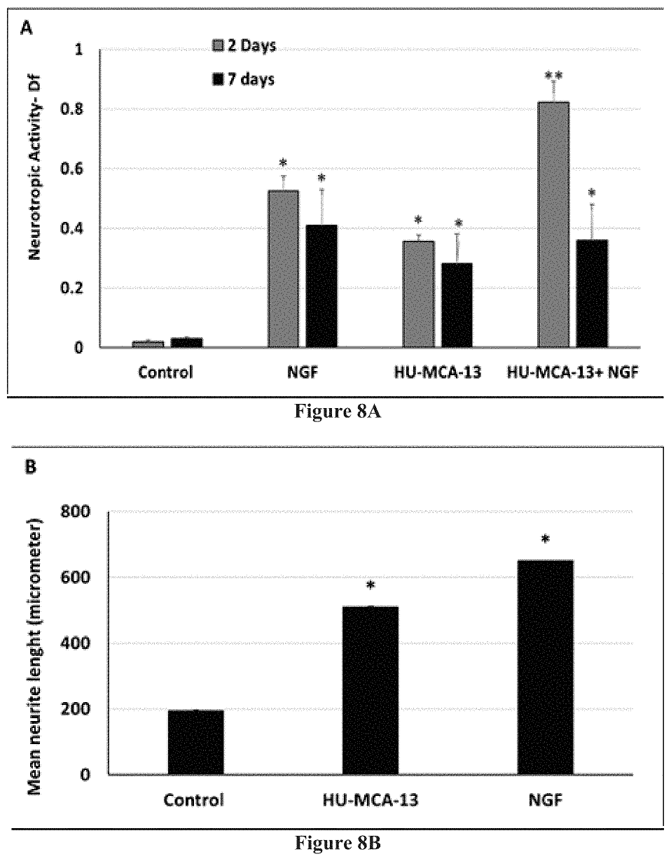

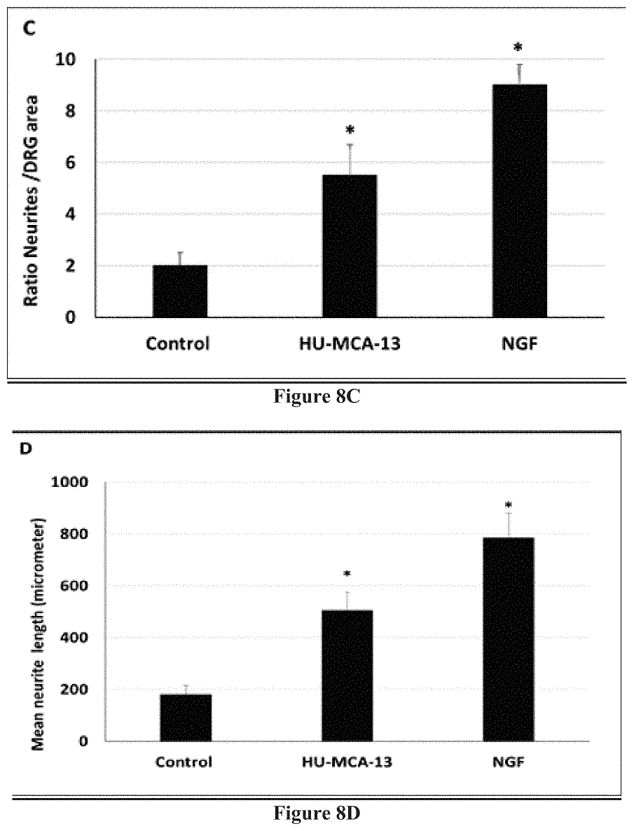

The results in FIGS. 6, 7 and 8 clearly indicate in the blank control medium there was very poor spontaneous neurite outgrowth of a length of about 200 micrometer and a low ration of 3 between the area covered by neurites and that covered by DRG ganglia. By contrast, NGF, the positive control, induced a strong, almost 3-4 fold increase in neurite outgrowth to a value of 700 micrometer and a ratio of 9 between the area covered by neurites and that covered by the DRG ganglia. Similarly, HU-MCA-13 morphologically, induced a strong neurite outgrowth, neurotropic effect like NGF; However, these neurites were less mature than in NGF-treated ganglia as evident from the lower expression of the cytoskeletal-tubulin protein staining (green photos). These findings may suggest that the maturation of the axons induced by HU-MCA-13 requires longer period of time than for NGF, an issue requiring further experimentation. These findings extend the neurotropic effects of HU-MCA-13 to DRG sensory neurons in addition to PC12 dopaminergic neuronal cultures.

HU-MCA-13 Induced Neurite Outgrowth in Adult Rat Spinal Cord Primary Cultures

To study the effect of HU-MCA-13 on adult rat spinal cord primary cultures in vitro, a purification procedure was developed that yields highly enriched motor neurons cultures. Spinal cord motor neuron cultures are an important tool for the study of mechanisms involved in motor neuron survival, degeneration and regeneration, motor neuron disorders such as amyotrophic lateral sclerosis or spinal muscular atrophy as well as in spinal cord injury. This bioassay was performed using vehicle (1% DMSO) DMEM negative control and 10 μM HU-MCA-13, tested compound. Briefly, dissected pieces of adult rat spinal cord tissue were maintained in phosphate-buffered saline (PBS) without Ca²⁺ and Mg²⁺ and pooled together and transferred to an enzymatic dissociation media containing 20 IU/ml papain in Earle's balanced salt solution (Worthington Biochemical, Freehold, N.J.) and incubated for 30 min at 37° C. After enzymatic dissociation, the papain solution was aspirated and the tissue was mechanically triturated with a fire-polished Pasteur pipette in complete media [Neurobasal Medium with B-27 supplement (Gibco, Grand Island, N.Y.), 100 IU/ml penicillin, 100 mg/ml streptomycin, 3.3 μg/ml aphidicolin, 0.5 mM glutamate] containing 2000 IU/ml DNase and 10-mg/ml protease inhibitors. Single-cell suspensions in complete media was plated on pre-coated collagen IV/laminin mixture-coated 96-well plates (Becton-Dickinson, Bedford, Mass.) at a density of 1.0×10⁴ cells/well. The spinal cord motor neurons (red arrows) and accessory Schwann cells (orange) were maintained for one day in culture and then exposed to 1% DMSO-DMEM negative control or 10 μM HU-MCA-13 for 7 days. The cultures were photographed, and Image analysis was performed. Neurite outgrowth was measured and quantified as for PC12 cultures, focusing on two parameters: a. SUM-total area covered by neurite outgrowths; b. mean length of a neurite (microns).

The results in FIGS. 6, 7 and 8 clearly indicate that in control medium there was very poor spontaneous neurite outgrowth of a total length of about 220 micrometer with a mean length of 150 micrometer of the neurite. By contrast, HU-MCA-13 induced a strong neurite outgrowth, four folds neurotropic effect, with a total (SUM) neurite length of 600 micrometer and a mean length of 350 micrometer of the neurite. These findings further extend the neurotropic effect of HU-MCA-13 to spinal cord motor neurons (cholinergic neurons), in addition to DRG neurons (peptidergic neurons) and PC12 cultures (dopaminergic neurons).

In summary, autonomic and sensor-motory tests indicate that HU-MCA-13, delivered by iv injection in a single-dose of 250 mg/kg, is acute-tolerated in mice. Furthermore, blood analysis findings further support acute tolerability of 250 mg/kg HU-MCA-13 upon iv injection in male C57BL/6 mice. A neurotropic effect was found between HU-MCA-13 and NGF in the PC12 dopaminergic neuronal model. HU-MCA-13 induced neurite outgrowth in dorsal root ganglion (DRG) sensory neurons. HU-MCA-13 induced neurite outgrowth in adult rat spinal cord primary cultures. These findings indicate safety of HU-MCA-13 and further extend the neurotropic effect of HU-MCA-13 to spinal cord motor neurons (cholinergic neurons), in addition to DRG neurons (peptidergic neurons) and PC12 cultures (dopaminergic neurons), justifying further drug development.

Synthesis

All compounds were prepared according to the general procedures in (a) Mostinski, Y.; Valerio, V.; Lankri, D.; Tsvelikhovsky, D. J. Org. Chem. 2015, 80, 10464-10473. (b) Valerio, V.; Mostinski, Y.; Kotikalapudi, R.; Tsvelikhovsky, D. Chem. Eur. J. 2016, 22, 2640-2647. (c) Albarghouti, G.; Kotikalapudi, R.; Lankri, D.; Valerio, V.; Tsvelikhovsky, D. Chem. Comm. 2016, 52, 3095-3098. As detailed in Scheme 2A-2B below.

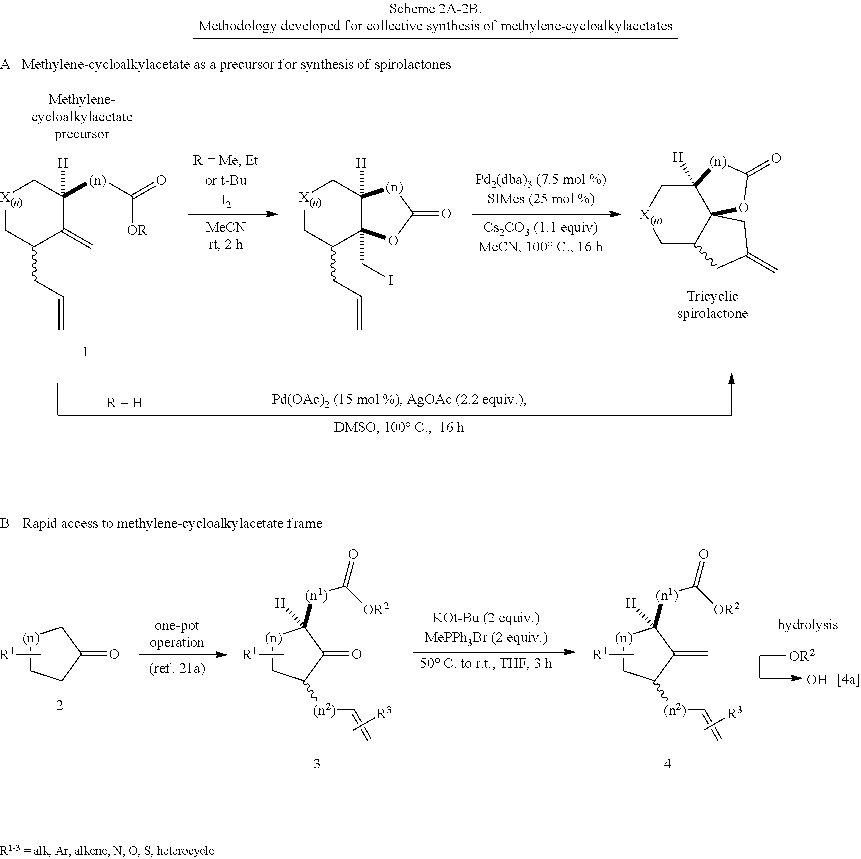

Methyl 2-(3-(2-methylallyl)-2-methylenecyclohexyl)acetate (5): 4.5 mmol of corresponding starting material (ketone 2; Scheme 2) were employed. Purification of the final crude product by flash column chromatography (5% ethyl acetate in hexane) yielded pure 5 (0.86 g, 87% yield, colorless oil) as mixture of diastereomers. ¹H NMR (300 MHz, CDCl₃): δ 4.82-4.52 (m, 4H), 3.67 (s, 3H), 2.69-2.41 (m, 2H), 2.38-2.23 (m, 2H), 2.17=1.95 (m, 2H), 1.94-1.82 (m, 2H), 1.82-1.73 (m, 1H), 1.70 (s, 3H), 1.64-1.41 (m, 1H), 1.34-0.76 (m, 2H). ¹³C NMR (75 MHz, CDCl₃): Major diastereomer: δ 173.6, 155.0, 144.1, 111.69, 101.8, 51.5, 41.2, 41.0, 41.0, 38.0, 35.5, 34.5, 26.0, 22.3. Minor diastereomer, characteristic peaks: δ 111.5, 105.9, 51.4, 40.9, 37.9, 37.6, 33.8, 32.3, 22.1, 21.1. IR (neat): 2924, 2853, 1739, 1643, 1437, 1164, 885 cm⁻¹.

Methyl 2-(3-(3-methylbut-2-en-1-yl)-2-methylenecyclohexyl)acetate (7): 9.7 mmol of corresponding starting material (ketone 2; Scheme 2) were employed. Purification of the final crude product by flash column chromatography (5% ethyl acetate in hexane) yielded pure 7 (1.9 g, 83% yield, colorless oil) as mixture of diastereomers. ¹′H NMR (300 MHz, CDCl₃): δ 5.13-5.05 (m, 1H), 4.59 (d, J=23.0 Hz, 2H), 3.65 (s, 3H), 2.65-2.56 (m, 1H), 2.48-2.36 (m, 1H), 2.33-2.16 (m, 2H), 2.01-1.81 (m, 4H), 1.78-1.71 (m, 1H), 1.68 (s, 3H), 1.59 (s, 3H), 1.52-1.21 (m, 1H), 1.12-0.82 (m, 2H). ¹³C NMR (75 MHz, CDCl₃): δ 173.6, 155.2, 132.0, 123.3, 101.8, 51.5, 44.4, 40.9, 38.0, 35.5, 34.7, 31.2, 26.1, 25.8, 17.9. IR (neat): 2922, 2853, 1739, 1435, 1164, 887 cm⁻¹.

Methyl 2-(3-allyl-2-methylene-5-phenylcyclohexyl)acetate (11): 1.4 mmol of corresponding starting material (ketone 2; Scheme 2) were employed. Purification of the crude product by flash column chromatography (5% ethyl acetate in hexane) yielded pure 11 (307 mg, 77% yield, colorless oil) as mixture of diastereomers. ¹′H NMR (300 MHz, CDCl₃): δ 7.35-7.12 (m, 5H), 5.93-5.66 (m, 1H), 5.16-4.89 (m, 2H), 4.82-4.60 (m, 2H), 3.68 (s, 3H), 3.15-2.25 (m, 6H), 2.23-1.61 (m, 4H), 1.34-1.15 (m, 1H). ¹³C NMR (75 MHz, CDCl₃): δ 173.4, 173.1, 153.8, 152.5, 145.9, 145.1, 137.4, 137.3, 137.1, 128.4, 128.4, 127.1, 126.9, 126.2, 126.2, 125.9, 116.2, 116.0, 115.7, 107.9, 107.7, 106.4, 102.7, 51.6, 51.6, 51.6, 45.2, 44.1, 43.2, 42.5, 41.9, 41.8, 40.8, 40.5, 39.5, 39.1, 39.0, 38.5, 38.4, 38.3, 38.3, 37.8, 37.8, 37.8, 37.5, 37.1, 36.9, 36.9, 35.0, 34.6. IR (neat): 2919, 2851, 1736, 1641, 1436, 1166, 893, 698 cm⁻¹.

Ethyl-3-allyl-5-(2-methoxy-2-oxoethyl)-4-methylenepiperidine-1-carboxylate (15): 5.1 mmol of corresponding starting material (ketone 2; Scheme 2) were employed. Purification of the final crude product by flash column chromatography (10% ethyl acetate in hexane) yielded pure 15 (447 mg, 31% yield, colorless oil) as mixture of diastereomers. ¹H NMR (300 MHz, CDCl₃): δ 5.87-5.69 (m, 1H), 5.11-5.01 (m, 2H), 4.82-4.70 (m, 2H), 4.17-3.96 (m, 4H), 3.67 (s, 3H), 2.70-2.41 (m, 4H), 2.39-2.26 (m, 2H), 2.21-1.95 (m, 2H), 1.23 (t, J=7.1 Hz, 3H). ¹³C NMR (75 MHz, CDCl₃): δ 172.4, 155.4, 150.3, 136.0, 116.8, 106.0, 61.5, 51.8, 50.2, 49.9, 42.1, 39.5, 34.5 (2C), 14.6. IR (neat): 980, 2913, 1738, 1726, 1643, 1434, 1231, 1159, 1126, 994, 897, 767 cm⁻¹.

Methyl 2-(3-(2-methylallyl)-2-oxocyclohexyl)acetate (18): 25.0 mmol of corresponding starting material (ketone 2; Scheme 2) were employed. Purification of the final crude product by flash column chromatography (10% ethyl acetate in hexane) yielded pure 18 (2.49 g, 44% yield, colorless oil) as mixture of diastereomers. ¹H NMR (300 MHz, CDCl₃): δ 4.75-4.55 (m, 2H), 3.63 (s, 3H), 2.94-2.82 (m, 1H), 2.79-2.68 (m, 1H), 2.57-2.44 (m, 2H), 2.18-2.05 (m, 3H), 1.90-1.72 (m, 3H), 1.64 (s, 3H), 1.44-1.12 (m, 2H). ¹³C NMR (75 MHz, CDCl₃): Major diastereomer: δ 211.6, 173.0, 143.3, 111.6, 51.6, 48.2, 47.4, 37.0, 34.9, 34.4, 34.2, 25.2, 22.4. Minor diastereomer, characteristic peaks: δ 43.6, 39.2, 36.9, 34.3, 30.7, 21.7, 20.1. IR (neat): 2934, 2858, 1736, 1709, 1436, 1165, 888 cm⁻¹.

Methyl 2-(3-allyl-2-oxocyclohexyl)acetate (20): 31.2 mmol of corresponding starting material (ketone 2; Scheme 2) were employed. Purification of the final crude product by flash column chromatography (5% ethyl acetate in hexane) yielded pure 20 (3.73 g, 57% yield, colorless oil) as mixture of diastereomers. ¹′H NMR (300 MHz, CDCl₃): δ 5.84-5.66 (m, 1H), 5.12-4.92 (m, 2H), 3.66 (s, 3H), 2.93-2.72 (m, 2H), 2.57-2.37 (m, 2H), 2.22-2.09 (m, 3H), 1.96-1.73 (m, 3H), 1.45-1.27 (m, 2H). ¹³C NMR (75 MHz, CDCl₃): δ 211.4, 173.0, 136.4, 116.2, 51.6, 50.1, 47.3, 34.8, 34.3, 34.1, 33.5, 25.1. IR (neat): 2932, 2859, 1736, 1708, 1436, 1199, 1166, 911 cm⁻¹.

Methyl 2-(3-(3-methylbut-2-en-1-yl)-2-oxocyclohexyl)acetate (21): 22.0 mmol of corresponding starting material (ketone 2; Scheme 2) were employed. Purification of the final crude product by flash column chromatography (10% ethyl acetate in hexane) yielded pure 21 (2.35 g, 44% yield, colorless oil) as mixture of diastereomers. ¹′H NMR (300 MHz, CDCl₃): δ 5.07-4.96 (m, 1H), 3.62 (s, 3H), 2.87-2.68 (m, 2H), 2.41-2.22 (m, 2H), 2.16-2.04 (m, 3H), 1.95-1.67 (m, 3H), 1.62 (s, 3H), 1.53 (s, 3H), 1.43-1.15 (m, 2H). ¹³C NMR (75 MHz, CDCl₃): δ 211.8, 173.1, 132.8, 122.0, 51.5, 51.0, 47.2, 34.8, 34.3, 34.2, 27.5, 25.7, 25.2, 17.7. IR (neat): 2928, 2857, 1737, 1709, 1436, 1173, 1109, 996, 857 cm⁻¹.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

### REFERENCES

1. (a) Sofroniew, M. V.; Howe, C. L.; Mobley, W. C. Annu. Rev. Neurosci. 2001, 24, 1217-1281. (b) Hennigan, A.; O'callaghan, R. M.; Kelly, A. M. neurodegeneration and neuroprotection, 2007, 424-427.
2. Xiao, N.; Le, Q. T. AITE, 2016, 64, 89-99.
3. (a) Faustino, C.; Rijo, P.; Reis, C. P. Pharmacol. Res. 2017, 120, 68-87. (b) Boado, R. J.; Zhang, Y.; Zhang, Y.; Pardridge, W. M. Biotechnol. Bioeng. 2007, 97, 1376-1386.
4. (a) Kuffler, D. P. Mol. Neurobiol. 1994, 9, 233-243. (b) Venkatesan, R.; Ji, E.; Kim, S. Y. BioMed Res. Int. vol. 2015, Article ID 814068, 22 pages, 2015. doi:10.1155/2015/814068.
5. More, S. V.; Koppula, S.; Kim, I. S.; Kumar, H.; Kim, B. W.; Choi, D. K. Molecules 2012, 17, 6728-6753.
6. (a) Mazzio, E.; Georges, B.; McTier, O.; Soliman, K. F. Neurochem. Res. 2015, 40, 2102-2112. (b) Ralets, I.; Østergaard, S.; Holm, A.; Kohler, L.; Bock, E.; Berezin, V. J. Neurosci. Methods 2004, 137, 61-69.
7. (a) Tabakman, R.; Lecht, S.; Lazarovici, P. BioEssays, 2004, 26, 80-90. (b) de los Rios, C.; Cano-Abad, M. F.; Villarroya, M.; López, M. G. Pflugers Arch. 2017, 1-12.
8. Greene, L. A.; Tischler, A. S. Proc. Natl. Acad. Sci. U.S.A. 1976, 73, 2424-2428.
9. Fujita, K.; Lazarovici, P.; Guroff, G. Environ. Health Perspect. 1989, 80, 127-142.
10. Vaudry, D.; Stork, P. J. S.; Lazarovici, P.; Eiden, L. E. Science, 2002, 296, 1648-1649.
11. Arien-Zakay, H.; Lecht, S.; Perets, A.; Roszell, B.; Lelkes, P. I.; Lazarovici, P. J. Mol. Neurosci. 2009, 37, 225-237.
12. Moosavi, F.; Hosseini, R.; Saso, L.; Firuzi, O. Drug Des. Dev. Ther. 2016, 10, 23-42.
13. Kubo, M.; Gima, M.; Baba, K.; Nakai, M.; Harada, K.; Suenaga, M.; Fukuyama, Y. Bioorganic Med. Chem. Lett., 2015, 25, 1586-1591.
14. Harada, K.; Kubo, M.; Horiuchi, H.; Ishii, A.; Esumi, T.; Hioki, H.; Fukuyama, Y. J. Org. Chem. 2015, 80, 7076-7088.
15. Chiu, S. P.; Wu, M. J.; Chen, P. Y.; Ho, Y. R.; Tai, M. H.; Ho, C. T.; Yen, J. H. J. Agric. Food Chem. 2012, 61, 9453-9463.
16. (a) Brown, G. D. Phytochemistry 1994, 36, 637-641. (b) Marcos, I. S.; Pedrero, A. B.; Sexmero, M. J.; Diez, D.; Garcia, N.; Basabe, P.; Urones, J. G. Synthesis 2005, 19, 3301-3310. (c) Marcos, I. S.; Pedrero, A. B.; Sexmero, M. J.; Diez, D.; Basabe, P.; García, N.; Urones, J. G. J. Org. Chem. 2003, 68, 7496-7504. (d) Marcos, I. S.; Hernndez, F. A.; Sexmero, M. J.; Diez, D.; Basabe, P.; Pedrero, A. B.; Urones, J. G. Tetrahedron Lett. 2002, 43, 1243-1245. (e) Marcos, I. S.; Hernndez, F. A.; Sexmero, M. J.; Diez, D.; Basabe, P.; Pedrero, A. B.; Urones, J. G. Tetrahedron 2003, 59, 685-694. (f) Marcos, I. S.; Pedrero, A. B.; Sexmero, M. J.; Diez, D.; Basabe, P.; Hernández, F. A.; Urones, J. G. Synlett. 2002, 01, 0105-0109. (g) Marcos, I. S.; Martinez, B.; Sexmero, M. J.; Diez, D.; Basabe, P.; Urones, J. G. Synthesis 2006, 22, 3865-3873.
17. Miyaoka, H.; Kajiwara, Y.; Yamada, Y. Tetrahedron Lett. 2000, 41, 911-914.
18. (a) Marcos, I. S.; Garcia, N.; Sexmero, M. J.; Basabe, P.; Diez, D.; Urones, J. G. Tetrahedron 2005, 61, 11672-11678. (b) Marcos, I. S.; Moro, R. F.; Basabe, P.; Diez, D.; Mollinedo, F.; Urones, J. G. Synlett. 2007, 13, 2017-2022.
19. Chiruvella, K. K.; Mohammed, A.; Dampuri, G.; Ghanta, R. G.; Raghavan, S. C. Int. J. Biomed. Sci. 2007, 3, 269-278.
20. (a) Piers, E.; Caillé, S.; Chen, G. Org. Lett. 2000, 2, 2483-2486. (b) Takahashi, M.; Dodo, K.; Hashimoto, Y.; Shirai, R. Tetrahedron Lett. 2000, 41, 2111-2114. (c) Corey, E. J.; Roberts, B. E. J. Am. Chem. Soc. 1997, 119, 12425-12431. (d) Jung, M. E.; Nishimura, N. Org. Lett. 2001, 3, 2113-2115. (e) Urones, L. G.; Teresa, J. D. P.; Marcos, L. S.; Martin, D. D.; Garrido, N. M.; Guerra, R. A. Phytochemistry 1987, 26, 1077-1079. (f) Marcos, I. S.; Conde, A.; Moro, R. F.; Basabe, P.; Díez, D.; Urones, J. G. Tetrahedron 2010, 66, 8280-8290. (g) Brown, G. D.; Liang, G. Y.; Sy, L. K. Phytochemistry 2003, 64, 303-323. (h) Sy, L. K.; Brown, G. D. Tetrahedron 2002, 58, 909-923. (i) Siva, B.; Poornima, B.; Venkanna, A.; Prasad, K. R.; Sridhar, B.; Nayak, V. L.; Babu, K. S. Phytochemistry 2014, 98, 174-182. (j) Connolly, J. D.; Thornton, I. M. S.; Taylor, D. A. H. J. Chem. Soc. 1970, 18, 1205-1205.
21. (a) Mostinski, Y.; Valerio, V.; Lankri, D.; Tsvelikhovsky, D. J. Org. Chem. 2015, 80, 10464-10473. (b) Valerio, V.; Mostinski, Y.; Kotikalapudi, R.; Tsvelikhovsky, D. Chem. Eur. J. 2016, 22, 2640-2647. (c) Albarghouti, G.; Kotikalapudi, R.; Lankri, D.; Valerio, V.; Tsvelikhovsky, D. Chem. Comm. 2016, 52, 3095-3098.
22. (a) Njar, V. C.; Adesanwo, J. K.; Raji, Y. Planta med. 1995, 61, 91-92. (b) Chiruvella, K. K.; Kari, V.; Choudhary, B.; Nambiar, M.; Ghanta, R. G.; Raghavan, S. C. FEBS lett. 2008, 582, 4066-4076. (c) Orisadipe, A.; Amos, S.; Adesomoju, A.; Binda, L.; Emeje, M.; Okogun, J.; Gamaniel, K. Biol. Pharm. Bull. 2001, 24, 364-367. (d) Marcos, I. S.; Pedrero, A. B.; Sexmero, M. J.; Diez, D.; Basabe, P.; Hernández, F. A.; Urones, J. G. Tetrahedron lett. 2003, 44, 369-372. (e) Marcos, I. S.; Moro, R. F.; Costales, I.; Basabe, P.; Diez, D.; Mollinedo, F.; Urones, J. G. Tetrahedron 2012, 68, 7932-7940. (f) Marcos, I. S.; Escola, M. A.; Moro, R. F.; Basabe, P.; Diez, D.; Sanz, F.; Urones, J. G. Bioorganic Med. Chem. 2007, 15, 5719-5737.
23. Sun, Y.; Wang, M.; Ren, Q.; Li, S.; Xu, J.; Ohizumi, Y.; Guo, Y. Fitoterapia 2014, 95, 229-233.
24. Chen, H. W.; Chen, L.; Li, B.; Yin, H. L.; Tian, Y.; Wang, Q.; Dong, J. X. Molecules 2013, 18, 14138-14147.
25. Tsang, C. K.; Kamei, Y. Eur. J. Pharmacol. 2004, 488, 11-18.
26. Oberbauer, E.; Urmann, C.; Steffenhagen, C.; Bieler, L.; Brunner, D.; Furtner, T.; Rivera, F. J. J. Nutr. Biochem. 2013, 24, 1953-1962.
27. Mena, M. A.; Davila, V.; Bogaluvsky, J.; Sulzer, D. Mol. Pharmacol. 1998, 54, 678-686.
28. Yang, C.; Zhao, J.; Cheng, Y.; Le, X. C.; Rong, J. ACS Chem. Neurosci. 2015, 6, 1560-1569.
29. Awaya, A.; Kobayashi, H.; Horikomi, K.; Tanaka, S.; Kabir, A. M.; Yokoyama, K.; Isayama, S. Biol. Pharm. Bull., 1993, 16, 248-253.
30. Kiryushko, D.; Kofoed, T.; Skladchikova, G.; Holm, A.; Berezin, V.; Bock, E. J. Bio. Chem. 2003, 278, 12325-12334.
31. Katzir, I.; Shani, J.; Regev, K.; Shabashov, D.; Lazarovici, P. J. Mol. Neurosci. 2002, 18, 251-264.
32. Arien-Zakay, H.; Lecht, S.; Perets, A.; Roszell, B.; Lelkes, P. I.; Lazarovici, P. J. Mol. Neurosci. 2009, 37, 225-237.
33. Momic, T.; Katzehendler, J.; Benny, O.; Lahiani, A.; Cohen, G.; Noy, E.; Lazarovici, P. J. Pharmacol. Exp. Ther. 2014, 350, 506-519.
34. Tobal et al. Molecules. 2019, 24(11), 1-26.

What is claimed is:

1. A compound having the general formula (I), including any stereoisomer or salt thereof: 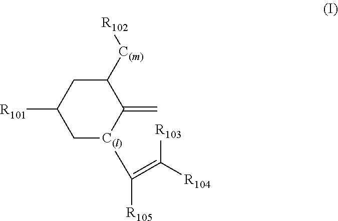
wherein
R₁₀₁ is selected from H, straight or branched C₁-C₁₀ alkyl, C₂-C₁₀ alkenyl, C₂-C₁₀ alkynyl;
m is an integer selected from 1-10;
—C₍ₘ₎— is selected from a straight or branched alkylene, straight or branched alkenylene, straight or branched alkynylene; optionally interrupted by at least one heteroatom;
R₁₀₂ is selected from —C(═O)R₁₀₈, —C(═S)R₁₀₉, —C(═P)R₁₁₀, —C(═CR₁₁₁R₁₁₂)R₁₁₃, straight or branched C₂-C₁₀ alkenyl, straight or branched C₂-C₁₀ alkynyl;
each of R₁₀₈, R₁₀₉, R₁₁₀, R₁₁₁, R₁₁₂ and R₁₁₃ are independently is selected from a group consisting of OH, —OR₁₁₄, —NH₂, —NHR₁₁₅, —NR₁₁₆R₁₁₇;
each of R₁₁₄, R₁₁₅, R₁₁₆ and R₁₁₇ are independently selected from straight or branched C₁-C₁₀ alkyl;
l is an integer selected from 1-10;
—C₍ₗ₎— is selected from a straight or branched alkylene, straight or branched alkenylene, straight or branched alkynylene; optionally interrupted by at least one heteroatom;
R₁₀₃, R₁₀₄, R₁₀₅ are each independently selected from H, straight or branched C₁-C₁₀ alkyl, C₂-C₁₀ alkenyl, C₂-C₁₀ alkynyl;
provided that when R₁₀₁ is H
(i) at least one of R₁₀₃, R₁₀₄ and R₁₀₅ is different than H; OR
(ii) m>1.

2. The compound according to claim 1, wherein R₁₀₂ is a straight or branched C₁-C₁₀ alkyl.

3. The compound according to claim 1, wherein R₁₀₂ is H.

4. The compound according to claim 1, wherein m is 1.

5. The compound according to claim 1, wherein m is 2-5.

6. The compound according to claim 1, wherein m is 2, l is 1 and R₁₀₂ is —C(═O)OH.

7. The compound according to claim 1, wherein R₁₀₁ is H.

8. The compound according to claim 1, wherein R₁₀₁ is a straight or branched C₁-C₁₀ alkyl.

9. The compound according to claim 1, wherein R₁₀₃ is a straight or branched C₁-C₁₀ alkyl.

10. The compound according to claim 1, wherein R₁₀₄ is a straight or branched C₁-C₁₀ alkyl.

11. The compound according to claim 1, wherein R₁₀₅ is a straight or branched C₁-C₁₀ alkyl.

12. The compound according to claim 1, wherein R₁₀₃, R₁₀₄, R₁₀₅ are each independently a straight or branched C₁-C₁₀ alkyl.

13. The compound according to claim 1, wherein R₁₀₃, R₁₀₄, R₁₀₅ are H.

14. The compound according to claim 1, wherein l is 1-5.

15. The compound according to claim 1, selected from the following:

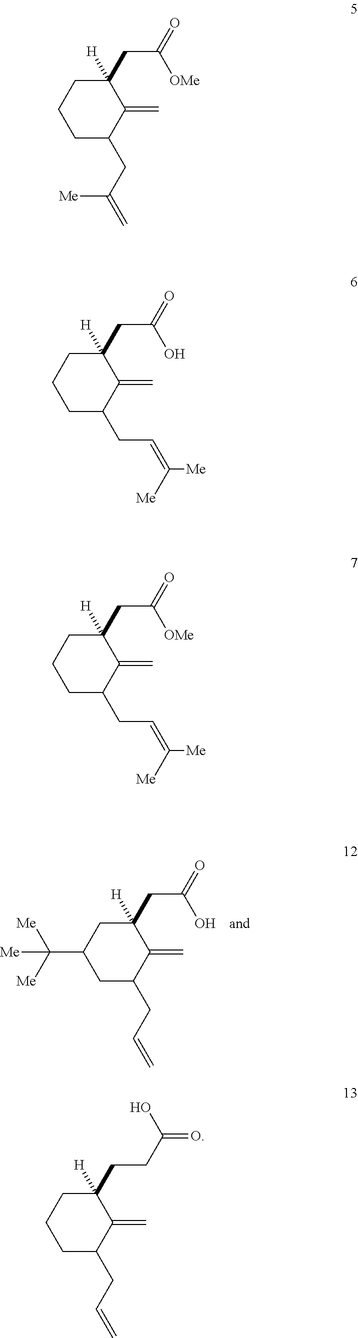

16. A method of treatment of a neurotropic condition in a subject in need thereof; said method comprising administering to said subject a compound of the general formula (II), including any stereoisomer or salt thereof: 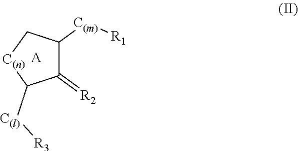
wherein
Ring A is optionally a saturated or unsaturated ring having optionally at least one heteroatom; and is optionally substituted by at least one group selected from straight or branched C₁-C₁₀ alkyl, straight or branched C₂-C₁₀ alkenyl, straight or branched C₂-C₁₀ alkynyl, CN, —OR₄, —NR₅R₆, —C(═O)R₇, halogen;
R₄, R₅ and R₆ are each independently selected from H, halogen, straight or branched C₁-C₁₀ alkyl, straight or branched C₂-C₁₀ alkenyl, straight or branched C₂-C₁₀ alkynyl;
R₇ is selected from H, straight or branched C₁-C₁₀ alkyl, straight or branched C₂-C₁₀ alkenyl, straight or branched C₂-C₁₀ alkynyl, halogen, OH, O(C₁-C₁₀)alkyl, NH₂, amine;
n is an integer selected from 1-10;
—C₍ₙ₎— is selected from a straight or branched alkylene, straight or branched alkenylene, straight or branched alkynylene; optionally interrupted by at least one heteroatom;
m is an integer selected from 1-10;
—C₍ₘ₎— is selected from a straight or branched alkylene, straight or branched alkenylene, straight or branched alkynylene; optionally interrupted by at least one heteroatom;
R₁ is selected from —C(═O)R₈, —C(═S)R₉, —C(═P)R₁₀, —C(═CR₁₁R₁₂)R₁₃, straight or branched C₂-C₁₀ alkenyl, straight or branched C₂-C₁₀ alkynyl;
each of R₈, R₉, R₁₀, R₁₁, R₁₂ and R₁₃ are independently is selected from a group consisting of OH, —OR₁₄, —NH₂, —NHR₁₅, —NR₁₆R₁₇;
each of R₁₄, R₁₅, R₁₆ and R₁₇ are independently selected from straight or branched C₁-C₁₀ alkyl;
R₂ is selected from O, S, CR₁₈R₁₉;
each of R₁₈ and R₁₉ is independently selected from H, straight or branched C₁-C₁₀ alkyl, halogen, CF₃SO₃, OH, C₁-C₁₀ alkoxy.
l is an integer selected from 1-10;
—C₍ₗ₎— is selected from a straight or branched alkylene, straight or branched alkenylene, straight or branched alkynylene; optionally interrupted by at least one heteroatom;
R₃ is selected from C(═O)R₂₀, OR₂₁, C(═O)OR₂₂, CF₃SO₃, straight or branched C₂-C₁₀ alkenyl, straight or branched C₂-C₁₀ alkynyl;
each of alkenyl or alkylenyl groups are optionally substituted by at least one group selected from C(═O)R₂₃, OR₂₄, halogen, CF₃SO₃;
each of R₂₀, R₂₁, R₂₂, R₂₃ and R₂₄ is independently selected from H, OH, halogen, straight or branched C₁-C₁₀ alkyl, straight or branched C₁-C₁₀ alkoxy, NH₂, amine.

17. A method according to claim 15, wherein said neurotropic condition is associated with neuronal cell death and/or loss of neuronal pathways.

18. A method according to claim 15, wherein said neurotropic condition benefits from mimicking Neuronal Growth Factor (NGF) activity.

19. A method according to claim 15, wherein said neurotropic condition is selected from Parkinson's disease, Alzheimer's disease, traumatic brain injuries, stroke, psychiatric disorders, choreas, ALS, conditions benefiting from neuronal regeneration, conditions benefiting from scaffolds for neural tracks regeneration, conditions benefiting from neural stem cell differentiation, conditions benefiting from tissue engineering, conditions benefiting from regenerative medicine and any combinations thereof.

20. A method according to claim 15, wherein said treatment provides peripheral nerve repair.
